(19) United States
(12) Patent Application Publication
(10) Pub. No.: US 2016/0151451 A1
(43) Pub. Date: Jun. 2, 2016

(54) METHOD FOR GENERATING CARDIOMYOCYTES

(71) Applicant: Korea Advanced Institute of Science and Technology (KAIST), Daejeon (KR)

(72) Inventors: Sung Woo CHO, Daejeon (KR); Seon Pyo Hong, Daejeon (KR); Sukhyun Song, Daejeon (KR); Yong-Mahn Han, Daejeon (KR); Gou Young Koh, Daejeon (KR)

(21) Appl. No.: 14/555,678

(22) Filed: Nov. 27, 2014

(51) Int. Cl. A61K 38/13; A61K 31/4409; C12N 5/077; A61K 45/06; A61K 35/34; A61K 31/355; A61K 31/444

(52) U.S. Cl. CPC A61K 38/13; A61K 31/355; A61K 31/4409; A61K 31/444; A61K 45/06; A61K 35/34; C12N 5/0657; C12N 2501/48; C12N 2501/727; C12N 2501/415

(57) ABSTRACT

The present application describes a method of creating cardioblasts and cardiomyocytes.

### BACKGROUND OF THE INVENTION

1. Field of the Invention

The present invention relates to the field of generating cardioblasts and differentiated cardiomyocytes from pluripotent cells, and uses of the cardiomyocytes to treat cardiac diseases.

2. General Background and State of the Art

The ability to generate specific cell types from pluripotent stem cells (PSCs) provides therapeutic strategies in cell-based therapy to rescue damaged organs (Passier et al., 2008). Since cardiac disease still remains as a leading cause of mortality worldwide and damaged cardiomyocytes cannot regenerate after myocardial injury, cardiac lineages are one of the most attractive cellular resources from PSCs (Burridge et al., 2012; Laflamme and Murry, 2011; Soonpaa et al., 2013). However, several major obstacles remain that hinder PSC-derived cardiac cell therapy from becoming a reliable and clinically applicable strategy for cardiac regeneration (Ban et al., 2013; Segers and Lee, 2008). Among these difficulties, obtaining a sufficient amount of purified cardiomyocytes from PSCs is the most important challenge and consideration in cell-based therapy. Although various methods for differentiating cardiomyocytes from PSCs have been developed, the cardiomyocyte differentiation process is very complex, and each individual step of the differentiation protocol must be precisely optimized (Burridge et al., 2012). Therefore, many researchers have concentrated their efforts on establishing improved strategies to efficiently induce cardiac lineage differentiation.

One of the most reliable approaches to date for efficient cardiomyocyte differentiation in vitro is the modulation of regulatory mechanisms that are fundamental in specifying different cell types during in vivo embryonic development (Burridge et al., 2012). Specifically, Flk1⁺ mesodermal precursor cells (MPCs) derived from differentiating PSCs were previously identified as cardiovascular progenitors both in vitro and in vivo, which can give rise to cardiomyocytes, endothelial cells, hematopoietic cells, and mural cells via multiple signaling pathways (Joo et al., 2012; Joo et al., 2011; Kattman et al., 2006; Yamashita et al., 2000; Yamashita et al., 2005; Yang et al., 2008). In order to restrict differentiation of PSCs strictly into cardiac lineages, various small molecules for cardiomyogenesis have been tested, most of which are related to signaling pathways, such as bone morphogenic protein (BMP), transforming growth factor (TGF), activin, nodal, Wnt, rho-associated coiled-coil kinase (ROCK), and fibroblast growth factor (FGF) (Burridge et al., 2012; Verma et al., 2013). However, each signaling pathway has no stringent role only for cardiomyogenesis, and a single signaling modulation cannot generate a sufficient amount of cardiomyocytes from PSCs for successful cardiac regeneration (Burridge et al., 2012). Thus, to overcome these hurdles in generating cardiomyocytes with high efficiency and reliability, we reported that Cyclosporine A (CsA) and antioxidants synergistically promote cardiomyocyte differentiation from Flk1⁺ MPCs by modulating the mitochondrial permeability transition pore (mPTP) and redox signaling (Cho et al., 2014; Fujiwara et al., 2011; Yan et al., 2009). Nevertheless, the current efficiency of cardiomyocyte differentiation requires significant improvement to yield clinically significant quantities of cardiomyocytes.

In the present study, we screened various signaling molecules and established a novel, simple, and efficient method for cardiomyocyte differentiation using a combination of such reagents—CsA, ROCK inhibitor Y27362, antioxidant Trolox, and activin A receptor type II-like kinase (ALK5) inhibitor EW7197 (hereafter referred to as “CsAYTE”)—which can generate highly enriched cardioblasts. Indeed, CsAYTE highly promoted the generation of a novel cardiac lineage-committed cell population, PDGFRα⁺Flk1⁻ cardioblasts (hereafter called as “PCBs”). Such CsAYTE-induced PCBs spontaneously further differentiated into functional αMHC⁺ cardiomyocytes (hereafter called as “M⁺CMs”). Importantly, although implantation of immature PCBs could not restore cardiac systolic function due to improper integration to the host myocardium, PCB-derived mature M⁺CMs faithfully restore cardiac systolic function through proper integration in a murine model of acute myocardial infarction (MI). These results provide compelling evidence and a novel approach to obtain an ample amount of cardioblasts, which can spontaneously and ultimately differentiate into functional cardiomyocytes for effective cardiac regeneration.

### SUMMARY OF THE INVENTION

Obtaining a sufficient amount of cardiomyocytes from pluripotent stem cells (PSCs) is one of the most difficult challenges and ultimate goals in cell-based therapy to rescue damaged hearts. Here, we show that CsAYTE, a combination of small molecules—Cyclosporine A (CsA), ROCK inhibitor Y27362, antioxidant Trolox, and ALK5 inhibitor EW7197—robustly generates into cardiobalsts from mouse and human PSCs. The cardioblasts are featured as PDGFRα⁺Flk1⁻ cardioblasts (PCBs), which we characterize as a proliferating population in a morphologically and functionally immature state. It was noted that the CsAYTE-induced PCBs spontaneously differentiated into functional αMHC⁺ cardiomyocytes (M⁺CMs) in regular medium without further treatment with CsAYTE and feeder cells. Importantly, PCB-derived M⁺CMs restore cardiac systolic function through proper integration to the host myocardium in a murine model of acute myocardial infarction. Taken together, an ample amount of PCBs can be obtained by the combined modulations of intracellular signaling and functional M⁺CMs derived from the PCBs hold great promise to rescue damaged hearts.

The present application discloses that CsAYTE efficiently generates PDGFRα⁺Flk1⁻ cardioblasts (PCBs) from PSCs. PCBs can be spontaneously differentiated into functional cardiomyocytes. PCBs are characterized as proliferating yet immature cardiac lineage-committed cells. And PCB-derived cardiomyocytes restore cardiac function in myocardial infarction model.

In one aspect, the present invention is directed to a method for inducing pluripotent cell to form cardioblast comprising contacting the pluripotent cell with an effective amount of a composition comprising inhibitor of mitochondrial permeability transition pore (mPTP), Rho-associated protein kinase (ROCK) inhibitor, antioxidant, or activin A receptor type II-like kinase (ALK5) inhibitor compound so as to form cardioblast.

In the method above, the composition may include any two, three or four of inhibitor of mitochondrial permeability transition pore (mPTP), ROCK inhibitor, antioxidant, or activin A receptor type II-like kinase (ALK5) inhibitor compound, and may optionally include Wnt signaling inhibitor compound.

In the method above, the inhibitor of mitochondrial permeability transition pore (mPTP) may be cyclosporine, in particular, Cyclosporine-A. The ROCK inhibitor may be RKI, RKI-II, siRNA-ROCK1, si-RNA-ROCK2 or chemical compound specific to ROCK1 or ROCK2. The chemical compound specific to ROCK1 or ROCK2 may be [(+)-(R)-trans-4-(1-aminoethyl)-N-(4-pyridyl) cyclohexanecarboxamide dihydrochloride] (Y27362). The antioxidant may be Trolox (6-hydroxy-2,5,7,8-tetramethylchroman-2-carboxylic acid). The activin A receptor type II-like kinase (ALK5) inhibitor may be N-[[4-([1,2,4]triazolo[1,5-a]pyridin-6-yl)-5-(6-methylpyridin-2-yl)-1H-imidazol-2-yl]methyl]-2-fluoroaniline (EW-7197). The method above may be conducted in vitro or in vivo. The pluripotent cell may be embryonic stem cell (ESC) or mesodermal stem cell (MSC). The pluripotent cell may be a mammalian cell, such as mouse or human. In the method above, phenotype for the mesodermal precursor cell may be Flk1⁺. Phenotype of the cardioblast may be characterized by PDGFRα⁺Flk1⁻.

In another aspect, the invention is directed to a method of generating cardiomyocytes comprising allowing the obtained cardioblast as above to proliferate with or without contact with the composition described above, to result in generation and expansion of cardiomyocytes.

In another aspect, the invention is directed to a method for generating and expanding differentiated cardiomyocytes comprising contacting a pluripotent cell with an effective amount of a composition comprising inhibitor of mitochondrial permeability transition pore (mPTP), Rho-associated protein kinase (ROCK) inhibitor, antioxidant, or activin A receptor type II-like kinase (ALK5) inhibitor compound so as to form cardiomyocyte.

In the method above, the inhibitor of mitochondrial permeability transition pore (mPTP) may be cyclosporine, in particular, Cyclosporine-A. The ROCK inhibitor may be RKI, RKI-II, siRNA-ROCK1, si-RNA-ROCK2 or chemical compound specific to ROCK1 or ROCK2. The chemical compound specific to ROCK1 or ROCK2 may be [(+)-(R)-trans-4-(1-aminoethyl)-N-(4-pyridyl) cyclohexanecarboxamide dihydrochloride] (Y27362). The antioxidant may be Trolox (6-hydroxy-2,5,7,8-tetramethylchroman-2-carboxylic acid). The activin A receptor type II-like kinase (ALK5) inhibitor may be N-[[4-([1,2,4]triazolo[1,5-a]pyridin-6-yl)-5-(6-methylpyridin-2-yl)-1H-imidazol-2-yl]methyl]-2-fluoroaniline (EW-7197). The method above may be conducted in vitro or in vivo. The pluripotent cell may be embryonic stem cell (ESC) or mesodermal stem cell (MSC). The pluripotent cell may be a mammalian cell, such as mouse or human. In the method above, phenotype for the mesodermal precursor cell may be Flk1⁺. Phenotype of the cardioblast may be characterized by PDGFRα⁺Flk1⁻. And optionally, contact of the composition described above with pluripotent cell may be withdrawn when cardioblast is formed.

In another aspect, the invention is directed to a method of regenerating a portion of a heart in a subject comprising:

(i) generating and expanding differentiated cardiomyocytes comprising contacting a pluripotent cell with an effective amount of a composition comprising inhibitor of mitochondrial permeability transition pore (mPTP), Rho-associated protein kinase (ROCK) inhibitor, antioxidant, or activin A receptor type II-like kinase (ALK5) inhibitor compound so as to form cardiomyocyte; and

(ii) administering to the subject the cardiomyocyte obtained in step (i) so as to expand cardiomyocytes in the heart. In this method, the heart may be damaged heart. And the damaged heart may be cardiomyopathy, myocardial infarction, acute myocardial infarction, chronic heart failure, ischemic and dilated cardiomyopathy, sick sinus syndrome, or congenital heart disease.

In the method above, the composition may include any two, three or four of inhibitor of mitochondrial permeability transition pore (mPTP), ROCK inhibitor, antioxidant, or activin A receptor type II-like kinase (ALK5) inhibitor compound, and may optionally include Wnt signaling inhibitor compound.

In the method above, the inhibitor of mitochondrial permeability transition pore (mPTP) may be cyclosporine, in particular, Cyclosporine-A. The ROCK inhibitor may be RKI, RKI-II, siRNA-ROCK1, si-RNA-ROCK2 or chemical compound specific to ROCK1 or ROCK2. The chemical compound specific to ROCK1 or ROCK2 may be [(+)-(R)-trans-4-(1-aminoethyl)-N-(4-pyridyl) cyclohexanecarboxamide dihydrochloride] (Y27362). The antioxidant may be Trolox (6-hydroxy-2,5,7,8-tetramethylchroman-2-carboxylic acid). The activin A receptor type II-like kinase (ALK5) inhibitor may be N-[[4-([1,2,4]triazolo[1,5-a]pyridin-6-yl)-5-(6-methylpyridin-2-yl)-1H-imidazol-2-yl]methyl]-2-fluoroaniline (EW-7197). The method above may be conducted in vitro or in vivo. The pluripotent cell may be embryonic stem cell (ESC) or mesodermal stem cell (MSC). The pluripotent cell may be a mammalian cell, such as mouse or human. In the method above, phenotype for the mesodermal precursor cell may be Flk1⁺. Phenotype of the cardioblast may be characterized by PDGFRα⁺Flk1⁻. And optionally, contact of the composition described above with pluripotent cell may be withdrawn when cardioblast is formed.

In another aspect, the invention is directed to a method of regenerating a portion of a heart in a subject comprising:

(i) generating and expanding cardioblast comprising contacting a pluripotent cell with an effective amount of a composition comprising inhibitor of mitochondrial permeability transition pore (mPTP), Rho-associated protein kinase (ROCK) inhibitor, antioxidant, or activin A receptor type II-like kinase (ALK5) inhibitor compound so as to form cardioblast; and

(ii) administering to the subject the cardioblast obtained in step (i) so as to differentiate into cardiomyocytes in the heart. In this method, the heart may be damaged heart. And the damaged heart may be cardiomyopathy, myocardial infarction, acute myocardial infarction, chronic heart failure, ischemic and dilated cardiomyopathy, sick sinus syndrome, or congenital heart disease.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1. Protocol for the differentiation of Flk1⁺MPCs into cardiomyocytes induced by four reagents in an OP9 co-culture system. LIF, Leukemia inhibitory factor.

FIGS. 2A-2D. Dose optimization of each reagent for cardiomyocyte differentiation. FIG. 2A and FIG. 2B show relative total cell numbers and percentages of cTnT⁺ cells with indicated concentrations of CsA, Y27632, Trolox and EW7197. Untreated cell population was regarded as 100%. Each group, n=3. *p<0.01 and #p<0.05 versus 0.

FIGS. 3A-3F. CsAYTE promotes differentiation into cardiomyocytes from mouse ESCs. (FIG. 3A and FIG. 3B) Representative FACS analysis and the percentage of mouse ESC-derived cTnT⁺ cells incubated with indicated molecules. Con, Control; Cs, CsA (3 μg/mL); Y, Y27632 (10 μM); T, Trolox (400 μM); E, EW7197 (1 μg/mL). Each group, n=4. (FIG. 3C and FIG. 3D) Images displaying α-actinin⁺ cells and DAPI⁺ nuclei, and the quantification analysis of α-actinin⁺ area (%). Each group, n=3. (FIG. 3E and FIG. 3F) Live cell images showing αMHC-GFP⁺ cells and comparison of αMHC-GFP⁺ area (%). Each group, n=4. In all graphs, *p<0.01 versus Con; #p<0.01 versus CsA. Scale bars, 100 μm.

FIGS. 4A-4B. CsAYTE promotes differentiation into cardiomyocytes from mouse ESCs in feeder free condition. (FIG. 4A and FIG. 4B) Representative FACS analysis and percentage of mouse ESC-derived cTnT⁺ cells grown in feeder-free culture. Each group, n=3. *p<0.01 versus Con; #p<0.01 versus CsA.

FIGS. 5A-5B. CsAYTE promotes differentiation into cardiomyocytes from mouse iPSCs. (FIG. 5A and FIG. 5B) Representative FACS analysis and percentage of mouse iPSC-derived cTnT⁺ cells grown in OP9 co-culture. Each group, n=4. *p<0.01 versus Con; #p<0.01 versus CsA.

FIGS. 6A-6D. CsAYTE promotes differentiation into cardiomyocytes from human iPSCs. (FIG. 6A and FIG. 6B) Representative FACS analysis and percentage of human iPSC-derived cTnT⁺ cells grown in feeder-free culture. Each group, n=3. (FIG. 6C and FIG. 6D) Images displaying human iPSC-derived cTnT⁺ cells, and the quantification analysis of cTnT⁺ area (%). Each group, n=3. In all graphs, *p<0.01 versus Con; #p<0.01 versus CsA. Scale bars, 100 μm.

FIGS. 7A-7B. CsAYTE generates cardioblasts that are differentiated into cardiomyocytes. (FIG. 7A) Protocol for generation of cardioblasts by CsAYTE. (FIG. 7B) Phase-contrast images showing differentiating Flk1⁺ MPCs incubated with a control vehicle (Control or Con), CsA (3 μg/mL) and CsAYTE. Scale bars, 100 μm.

FIGS. 8A-8B. The differentiation process of cardioblasts from Flk1⁺ MPCs. (FIG. 8A) Time-lapse images of differentiating Flk1⁺ MPCs incubated with CsAYTE. Interval, 10 hours (hr). Scale bars, 100 (FIG. 8B) Live cell image, which is magnified view of boxed region of (FIG. 8A), showing αMHC-GFP⁺expression during Flk1⁺ MPC differentiation incubated with CsAYTE. Scale bars, 50 μm.

FIGS. 9A-9C. CsAYTE generates PCBs from mouse ESC-derived Flk1⁺ MPCs. (FIG. 9A and FIG. 9B) Representative FACS analysis and percentage of PDGFRα⁺Flk1⁻ cells incubated with Control, CsA, and CsAYTE. Each group, n=4. *p<0.01 versus Con; #p<0.01 versus CsA. (FIG. 9C) Images of expressions of Flk1, in the PDGFRα⁺ cells of differentiating Flk1⁺ MPCs incubated with Control, CsA and CsAYTE. Scale bars, 100 μm.

FIGS. 10A-10B. CsAYTE generates PCBs from mouse iPSC-derived Flk1⁺ MPCs. (FIG. 10A and FIG. 10B) Representative FACS analysis and the percentage of mouse iPSC-derived PDGFRα⁺Flk1⁻ cells incubated with control vehicle (Con), CsA, and CsAYTE. Each group, n=4. *p<0.01 versus Con; #p<0.01 versus CsA.

FIGS. 11A-11B. CsAYTE highly induces PCBs compared to other reagents. (FIG. 11A and FIG. 11B) Representative FACS analysis and the percentage of PDGFRα⁺Flk1⁻ cells incubated with the indicated reagents. Each group, n=5. *p<0.01 versus Con.

FIGS. 12A-12B. CsAYTE induces cardiac lineage specification. (FIG. 12A and FIG. 12B) Images of expressions of Flk1, Nkx2.5 and cTnT in the PDGFRα⁺ cells of differentiating Flk1⁺ MPCs incubated with Control, CsA and CsAYTE. Scale bars, 100 μm.

FIGS. 13A-13F. CsAYTE reduces other mesodermal lineage cells. (FIG. 13A, FIG. 13B and FIG. 13C) Representative FACS analysis of PDGFRα and CD31 expression and representative FACS analysis and the percentage of CD144⁺CD31⁺ endothelial cells incubated with Control, CsA (3 μg/mL) and CsAYTE. Each group, n=4. *p<0.01 versus Con. (FIG. 13D, FIG. 13E and FIG. 13F) Representative FACS analysis of PDGFRα and CD41 expression and representative FACS analysis and the percentage of CD41⁺ early hematopoietic cells incubated with Control (Con), CsA (3 μg/mL) and CsAYTE. Each group, n=4. *p<0.01 versus Con.

FIGS. 14A-14C. PCB can spontaneously differentiated into functional cardiomyocytes without CsAYTE. (FIG. 14A) Protocol for the analysis of PCB-derived cardiomyocyte differentiation incubated with indicated reagents in OP9 co-culture system. (FIG. 14B and FIG. 14C) The percentage of cTnT⁺ and αMHC-GFP⁺ cells incubated with indicated reagents. Each group, n=3. *p<0.01, #p<0.05, and NS versus Con.

FIGS. 15A-15C. PCB can spontaneously differentiated into functional cardiomyocytes without CsAYTE and OP9 feeder cells. (FIG. 15A) Protocol for analysis of PCB-derived cardiomyocyte differentiation in feeder free culture. (FIG. 15B and FIG. 15C) Representative FACS analysis of PCB-derived cTnT⁺ cells and αMHC-GFP⁺ cells grown in feeder free culture.

FIGS. 16A-16D. PDGFRα⁺ cardioblasts transiently exist in myocardium during embryonic heart development. (FIG. 16A and FIG. 16B) Images showing α-actinin⁺ and PDGFRα⁺ cells and DAPI⁺ nuclei in the heart during embryonic development. Scale bars represent 100 μm (FIG. 16A) and 50 μm (FIG. 16B), respectively. (FIG. 16C) Quantification of PDGFRα⁺ area (%) during embryonic heart development in time dependent manner. Each group, n=3. *p<0.01 versus E9.5 (FIG. 16D) Time dependent-curves of the percentages of PCBs and cTnT⁺ cells incubated with CsAYTE. Each group, n=4.

FIGS. 17A-17C. Lineage tracing of PDGFRα⁺ cardioblasts in myocardium during embryonic heart development. (FIG. 17A) Protocol of tamoxifen injection to the PDGFRα-Cre^{ERT2}/tdTomato mouse in pregnancy for lineage tracing of PDGFRα expressing cells in embryonic heart. (FIG. 17B and FIG. 17C) Images showing PDGFRα⁺ tdTomato cells, α-actinin⁺ cardiomyocytes, and DAPI⁺ nuclei in E12.5 embryonic heart. Scale bars represent 100 μm (FIG. 17B) and 50 μm (FIG. 17C), respectively.

FIGS. 18A-18B. Exclusion of OP9 feeder cells from Flk1⁺ MPCs. (FIG. 18A) Representative FACS analysis of PDGFRα expression incubated with Control (Con), CsA (3 μg/mL) and CsAYTE. (FIG. 18B) Representative FACS analysis of OP9 feeder cells with and without Flk1⁺ MPCs.

FIG. 19. Protocol for generations and analyses of PCB and αMHC-GFP⁺ cardiomyocytes (M⁺CM).

FIGS. 20A-20C. PCB have more proliferative capacity than M⁺CM. (FIG. 20A and FIG. 20B) Representative FACS analysis for BrdU incorporation and the percentage of BrdU⁺ cells in PCB and M⁺CM. Each group, n=3. *p<0.01 versus PCB. (FIG. 20C) Relative mRNA expression levels of connexin43 gap junction in PCB and M⁺CM. Each group, n=3. *p<0.01 versus PCB.

FIGS. 21A-21B. Action potentials of M⁺CM. (FIG. 21A and FIG. 21B) 3 different types (nodal, atrial, and ventricular type) of action potentials and percentile distribution in M⁺CM. Dotted lines indicate zero voltage level.

FIGS. 22A-22C. Ion currents of M⁺CMs. (FIG. 22A) Delayed rectifying K⁺ current (I_{K}) evoked by depolarizing test pulses between −100 and +80 mV in 10-mV increment from a holding potential of −80 mV at 5 second interval and inhibited by TEA at test potentials between +50 and +80 mV. (FIG. 22B) Na⁺ current (I_{Na}) evoked by depolarizing test pulses between −70 mV and +50 mV in 10-mV increment from a holding potential of −80 mV at 5 second interval and inhibited by TTX at test potentials between −50 and −40 mV. (FIG. 22C) T-type Ca²⁺ current (I_{CaT}) activated by depolarizing test pulses between −60 and +50 mV in 10-mV increment from a holding potential of −40 mV at 5 s interval and inhibited by mibefradil at test potentials between −30 and +10 mV.

FIG. 23. Western blot analysis of ion channel protein expression during cardiomyocyte differentiation from ESCs.

FIGS. 24A-24E. PCBs are in a morphologically immature state. (FIG. 24A, FIG. 24B and FIG. 24C) Images showing Mitotracker⁺ mitochondria, cTnT⁺ sarcomere and DAPI⁺ nuclei and comparisons of Mitotracker⁺ and cTnT⁺ areas in PCB and M⁺CM. Scale bars, 20 μm. Each group, n=6. *p<0.01 versus PCB. (FIG. 24D and FIG. 24E) Transmission electron microscope images showing the mitochondrial morphology and cristae (white arrow heads) and quantification of mitochondrial size in PCB and M⁺CM. Scale bars, 500 nm. Each group, n=8. *p<0.05 versus PCB.

FIG. 25. Protocol for gene expression analyses of PCB with ESC, Flk1⁺ MPC, PDGFRα⁻Flk1⁻ cell, and M⁺CM.

FIGS. 26A-26B. PCBs are at an intermediate state between MPCs and differentiated cardiomyocytes. (FIG. 26A and FIG. 26B) Relative mRNA expression levels of pluripotency (oct4, nanog and sox2), mesoderm (mesp1, brachyury), cardiac transcription factor (nkx2.5, tbx5, isl1, gata4, and hand2), cardiac sarcomere protein (tnnt2 and myl7) and mitochondrial biogenesis (pgc1α) related genes in the indicated cells.

FIGS. 27A-27B. Protocol for microarray analysis. (FIG. 27A) Comparison and sampling for microarray analysis of Flk1⁺ MPC, PCB, PCB without CsAYTE (PCB-WOC) and M⁺CM. (FIG. 27B) Microarray gene expression heat map of whole genes. Red and green colors represent up- and down-regulations.

FIG. 28A and FIG. 28B show scatter plot and gene ontology analysis comparing PCB and PCB-WOC. In the graph, lines indicate 30 fold changes of transcripts between two groups and up- and down-regulated genes are shown in red and green colors, respectively.

FIG. 29A and FIG. 29B show scatter plot and gene ontology analysis comparing PCB and Flk1⁺ MPC. In the graph, lines indicate 30 fold changes of transcripts between two groups and up- and down-regulated genes are shown in red and green colors, respectively.

FIG. 30A and FIG. 30B show scatter plot and gene ontology analysis of comparing M⁺CM and PCB. In the graph, lines indicate 30 fold changes of transcripts between two groups and up- and down-regulated genes are shown in red and green colors, respectively.

FIG. 31. Experimental scheme of implantation of PCBs and M⁺CMs into acute MI murine model.

FIGS. 32A-32E. Generation of E14Tg2a ESC line with tdTomato fluorescence for tracing PCBs after implantation. (FIG. 32A, FIG. 32B, FIG. 32C, FIG. 32D, and FIG. 32E) Live cell images and FACS analysis showing tdTomato fluorescence from E14Tg2a ESC line, differentiating E14Tg2a ESCs and differentiating Flk1⁺ MPCs with OP9 feeder cells. Scale bars, 100 μm.

FIG. 33. Implantation of M⁺CMs, but not of PCBs, exhibits for functional recovery in the infarcted heart by MRI. End systolic and diastolic axial views of ventricular chambers in cardiac MRI and left ventricular chamber area during end systole (dotted line in red) of control, MI⁰, MI+M⁺CM, and MI+PCB.

FIGS. 34A-34D. Implantation of M⁺CMs, but not of PCBs, exhibits for functional recovery in the infarcted heart by TTE. (FIG. 34A, FIG. 34B, FIG. 34C, and FIG. 34D) M-mode TTE views of control, MI⁰, MI+M⁺CM, and MI+PCB and improved left ventricular wall motion in MI+M⁺CM (white arrow heads) and quantification of left ventricular internal dimension in systole (mm), ejection fraction (%), and fractional shortening (%).*p<0.01, Con versus MI⁰; #p<0.01, MI⁰ versus MI+M⁺CM; NS (not significant), MI⁰ versus MI+PCB.

FIGS. 35A-35B. Implantation of M⁺CMs, but not of PCBs, exhibits for structural recovery in the infarcted heart. (FIG. 35A) Gross images of hearts in control, MI⁰, MI+M⁺CM, and MI+PCB. Scale bars, 2.5 mm. (FIG. 35B) H&E staining of mid-sectioned hearts of control, MI⁰, MI+M⁺CM, and MI+PCB. Black arrows indicate tissue defects. 1 mm and 50 μm in the upper and lower panels, respectively.

FIGS. 36A-36C. Implanted M⁺CMs, but not PCBs, are integrated to host myocardium (FIG. 36A and FIG. 36B) Images showing engraftment of M⁺CMs and PCBs having tdTomato to the α-actinin⁺ host myocardium and formation of connexin43 gap junction (white arrow heads). Scale bars represent 1 mm in the upper panel, 50 μm in the left lower panel, and 10 μm in the right lower panel. (FIG. 36C) Scanning electron microscopy images showing engraftment of M⁺CMs and PCBs to the host myocardium and formation of junction (white arrow heads). Scale bars, 5 μm.

FIGS. 37A-37B. Schematic diagram of efficient cardioblast and cardiomyocyte differentiation from PSCs for cardiac regeneration. (FIG. 37A and FIG. 37B) Schematic diagram showing that CsAYTE promotes the differentiation of functional cardiomyocytes through expansion of PCBs by 22-folds compared to control. Moreover, implantation of PCB-derived functional cardiomyocytes, not immature PCBs, has a regenerative capacity in infarcted heart.

### DETAILED DESCRIPTION OF THE PREFERRED EMBODIMENTS

In the present application, “a” and “an” are used to refer to both single and a plurality of objects.

As used herein, “carriers” include pharmaceutically acceptable carriers, excipients, or stabilizers, which are nontoxic to the cell or mammal being exposed thereto at the dosages and concentrations employed. Often the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

As used herein, a “dose” refers to a specified quantity of a therapeutic agent prescribed to be taken at one time or at stated intervals.

As used herein, “effective amount” is an amount sufficient to effect beneficial or desired clinical or biochemical results. An effective amount can be administered one or more times. For purposes of this invention, an effective amount of a compound or cells is an amount that is sufficient to palliate, ameliorate, stabilize, reverse, slow or delay the progression of the disease state.

As used herein, “pluripotent cell” refers to refers to stem cells that can differentiate to all three germlines, endoderm, ectoderm and mesoderm, to differentiate into any cell type in the body, but cannot give rise to a complete organism. A totipotent stem cell is one that can differentiate or mature into a complete organism such as a human being.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment.

As used herein, “inhibitor of mitochondrial permeability transition pore (mPTP)” refers to any inhibitor of mPTP in the mitochondria of differentiating mesodermal precursor cells, such as any specific agent against this target. Such an inhibitor substance may include without limitation, NIM811, which is a mitochondrial permeability transition inhibitor and is also known as N-methyl-4-isoleucine cyclosporine, a four-substituted cyclosporine analogue that binds to cyclophilin, or Cyclosporine A. In a preferred embodiment, the inhibitor is without limitation, Cyclosporine A.

The meaning of “Rho-associated protein kinase (ROCK) inhibitor” is well known in the art. An assay for determining a ROCK suppressor or ROCK inhibitor can include for example the following method. ROCKs were discovered as the first effectors of Rho and they induce the formation of stress fibers and focal adhesions by phosphorylating MLC (myosin light chain). Therefore, an assay for determining ROCK suppression is confirming MLC phosphorylation inhibition such as by Western blotting. In this regard, a ROCK suppressor, Y27632 inhibits MLC phosphorylation in Flk1⁺ MPCs during differentiation.

Y27632 has the following chemical structure:

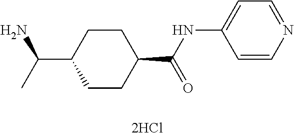 [SMILES: C[C@@H](N)[C@H]1CC[C@H](C(=O)Nc2ccncc2)CC1]

As used herein, the term “antioxidant” is well known in the art, and may include such a compound as N-acetyl-cystine, ascorbic acid or Trolox. In a preferred embodiment, the antioxidant is without limitation, Trolox.

As used herein, “activin A receptor type II-like kinase (ALK5) inhibitor” may include without limitation, compounds EW-7197 or 4-[4-(3,4-Methylenedioxyphenyl)-5-(2-pyridyl)-1H-imidazol-2-yl]benzamide (SB431542). In a preferred embodiment, the compound is without limitation, EW-7197.

As used herein, “cardiac damage” includes but is not limited to cardiomyopathy, myocardial infarction, acute myocardial infarction, chronic heart failure, ischemic and dilated cardiomyopathy, sick sinus syndrome, congenital heart disease and so forth where regeneration of a portion of the heart will treat the condition, where the implantation of functional cardiomyocytes regenerates a portion of the heart organ and thus reversing the damage to the heart.

As used herein, “functional cardiomyocytes” are those cardiomyocytes, when used on injured heart, results in functional recovery of infarcted heart. Preferably such cardiomyocytes are αMHC⁺ cardiomyocytes (hereafter called as “M⁺CMs”).

The present application discloses that contact of pluripotent stem cells with CsAYTE results in the induction of cardioblasts, which spontaneously, without necessarily further contact with CsAYTE, results in the differentiation of the cardioblasts to cardiomyocytes. Although this process is discussed in the context of creating the cardiomyocytes in vitro and implanting or injecting them to the site of injury in the subject, it can be also envisioned that the CsAYTE may be directly delivered to the site of injury in vivo together with the pluripotent stem cells so as to form the cardioblast and cardimyocytes in vivo.

Cardiomyocyte Production

Obtaining a sufficient amount of cardiomyocytes from PSCs is the most difficult and critical obstacle to utilizing stem cell therapy for cardiac regeneration (Burridge et al., 2012; Segers and Lee, 2008). Previously established methods for the differentiation of cardiomyocytes from PSCs in vitro require not only precise manipulation of timing and signaling molecules, but also more efficient and simple procedures for practical benefits (Burridge et al., 2012). Here, we developed a method to robustly promote cardiomyocyte differentiation using CsAYTE. With CsAYTE treatment, it is possible to generate differentiated populations that are highly enriched in cardiomyocytes without the need for cell sorting or genetic modification. Our method is quick, simple, and highly effective, compared to previous protocols (Cao et al., 2012; Kattman et al., 2011), as CsAYTE treatment alone is sufficient to generate functional cardiomyocytes after mesodermal induction. Most importantly, we can easily detect and follow the differentiation process at a single cell level on a feeder cell layer (Mummery et al., 2012; Yamashita et al., 2005), which made it possible for us to discover the novel PCBs after CsAYTE treatment.

Previous reports showed that PDGFRα is known as the cardiac mesodermal marker to segregate Flk1⁺ mesoderm into PDGFRα⁺Flk1⁺ cardiac and PDGFRα⁻Flk1⁺ hematopoietic subpopulations (Kattman et al., 2011; Liu et al., 2012). Compared to PDGFRα⁺Flk1⁺ cells, PCBs arise at a later stage during cardiomyocyte differentiation from Flk1⁺ MPCs. Although PDGFRα⁺Flk1⁺ cells are known as cardiac mesoderm, they are not efficient in differentiating into cardiomyocytes unless activin A and BMP4 levels are finely modulated (Kattman et al., 2011), while our data show that the PCBs can spontaneously further differentiate into cardiomyocytes without additional manipulation and stimulation. Furthermore, we also demonstrated that PDGFRα⁺ cardioblasts transiently exist in the embryonic myocardium at E8.5-9.5 during in vivo heart development. This is the first study demonstrating the existence and characteristics of cardioblasts during cardiomyogenesis.

To date, it remains to be determined what characteristics define differentiated cells as functionally implantable cardiomyocytes. Recently, functional attributes such as firing action potentials or oscillating calcium and global transcriptome analysis have been reported as additional hallmarks of induced cardiomyocytes (Addis and Epstein, 2013). In this aspect, we define PCBs as proliferating cardiac lineage-committed cells still in an intermediate state between MPCs and cardiomyocytes using genome wide, morphologic, and functional analyses. Specifically, PCBs showed less developed mitochondria and cardiac sarcomere structures and did not show any electrophysiological activities. In addition, previously reported cardiac progenitors, such as Nkx2.5⁺ reporter cells, were most abundant at days 8-10 after PSC differentiation induction (Christoforou et al., 2008; Elliott et al., 2011). In this study, PCBs appeared at day 5-6, which is a significantly earlier stage than that for previously known cardiac progenitors. Therefore, we can define and characterize the PCBs as a unique cardiac lineage cell population, which differs in differentiation stages and immuno-phenotypes from previously known cardiac progenitors.

Most importantly, we can generate large quantities of functional cardiomyocytes with CsAYTE treatment, which stimulates the expansion of PCBs, to restore cardiac systolic function in a murine model of acute MI. So far, numerous researchers and clinicians are investigating the optimal cellular resource derived from various types of stem cells for the functional recovery of a damaged heart (Garbern and Lee, 2013). Previous studies showed that the implantation of adult stem cell-derived non-cardiac cells, such as skeletal muscle progenitors, bone marrow-derived cells, and adipose tissue-derived cells, have minor effects on the improvement of cardiac function after MI (Burridge et al., 2012; Loffredo et al., 2011). Since these non-cardiac cell populations ultimately cannot replenish the loss of cardiomyocytes and their function, minor improvement in cardiac function is due to paracrine effects rather than direct effects. While the implantation of ESCs and iPSC-derived cardiac progenitors, or cardiomyocytes showed improvement of cardiac function after MI through direct integration, it is unclear which stage and state of cardiac lineage cells from PSCs were effective for cardiac regeneration (Chong et al., 2014; Christoforou et al., 2010; Kawamura et al., 2012; Shiba et al., 2012). A recently developed strategy, direct reprogramming, was used to generate cardiomyocytes from cardiac fibroblasts or other adult cells using cardiac-specific transcription factors (Gata4, Mef2c, and Tbx5) both in vitro and in vivo, and these reprogrammed cells were shown to have regenerative potential in the MI model (Ieda et al., 2010; Qian et al., 2012; Song et al., 2012). However, a conflicting report showed that these induced cardiomyocytes by direct reprogramming did not show any regenerative potential after their implantation into the injured heart due to incomplete acquirement of characteristic cardiomyocyte traits, including electrophysiology (Chen et al., 2012). The PCBs characterized in our study exhibit similar features, especially in their inability to contribute to cardiac regeneration, but these immature cells can be spontaneously and efficiently differentiated into highly therapeutic functional cardiomyocytes. Therefore, previous reports and our findings emphasize the importance of morphologically and functionally mature cardiomyocytes, which significantly reflect cardiac regenerative potential.

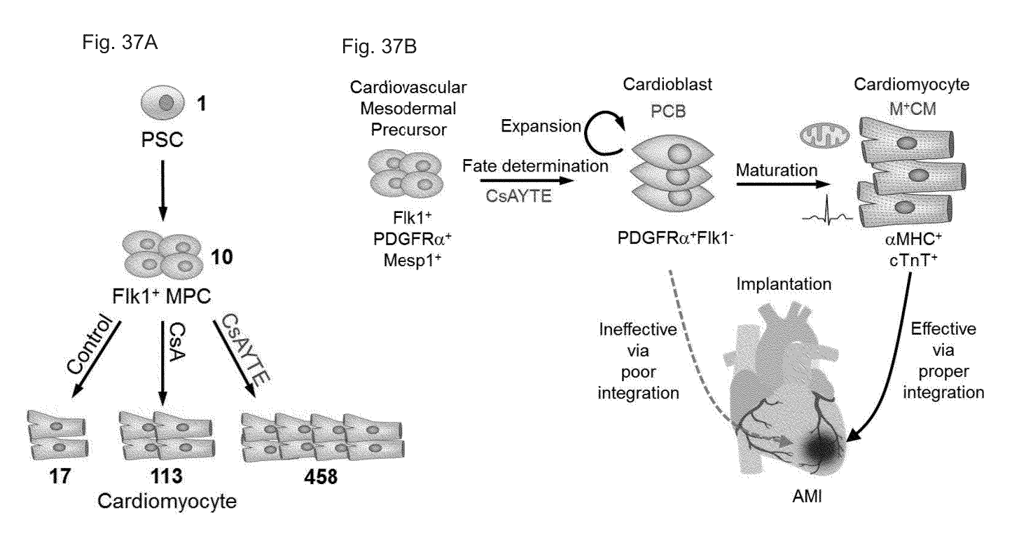

In summary, we have established a novel, efficient and convenient method to generate large quantities of cardioblasts, namely “PCB” using CsAYTE, which is a combination of four specific reagents that significantly enhance commitment of cardiac linage cells from the Flk1⁺ MPCs during differentiation (FIG. 37A). Moreover, functional cardiomyocytes can be derived from the increased PCB population with high efficiency and serve as the optimal cellular resource for cardiac regeneration (FIG. 37B). Taken together, our findings provide technical and conceptual advances for cardiac stem cell therapy in the field of cardiac regeneration.

Administration and Dosage

When administered therapeutically, the cardiomyocytes or cardioblasts of the invention such as M⁺CM, PCB, will result in regeneration of at least a portion of the heart organ. Associated proteins, chemicals or cells may be additionally administered in therapeutically effective amounts. In general, a therapeutically effective amount means that amount necessary to delay the onset of, inhibit the progression of, or halt altogether the particular condition being treated. Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication.

The agent of the invention should be administered for a length of time sufficient to provide either or both therapeutic and prophylactic benefit to the subject. Generally, the agent is administered for at least one day. In some instances, the agent may be administered for the remainder of the subject's life. The rate at which the agent is administered may vary depending upon the needs of the subject and the mode of administration.

The therapeutic agent may be administered in alone or in combination with other agents including proteins, receptors, co-receptors and/or genetic material designed to introduce into, upregulate or down regulate these genes in the area or in the cells.

Methods of Cardioblast or Cardiomyocyte Delivery to Heart

Enough cells should be implanted to the myocardium at the site of injury or infarction to maximize restoration of function. The cells may be administered via transvascular route. Cells can be infused directly into the coronary arteries and have a greater likelihood of remaining in the injured myocardium as a result of the activation of adhesion molecules and chemokines. Cells may be also injected intravenously. Alternatively, cells may be directly injected into the ventricular wall. In this regard, a transendocardial approach can be used in which a needle catheter is advanced across the aortic valve and positioned against the endocardial surface. Cells can then be injected directly into the left ventricle. Electrophysiological mapping can be used to differentiate sites of viable, ischemic, or scarred myocardium. In a transepicardial approach, cells are injected during open heart surgery. The advantage of this approach is that it allows direct visualization of the myocardium and easier identification of regions of scar and border zones of infarcted tissues. A third approach involves the delivery of cells through one of the cardiac veins directly into the myocardium.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Experimental Procedures

### Example 1.1

### PSC and OP9 Cell Culture

EMG7 mouse ESCs, which have α-MHC promoter-driven enhanced GFP gene, E14Tg2a ESCs and OP9 cells were generated and maintained as described previously (Hirai et al., 2003; Kodama et al., 1994; Yamashita et al., 2005). Mouse iPSCs derived from FVB strain were a generous gift from Drs. Hyun-Jai Cho and Hyo-Soo Kim (Seoul National University Hospital) and prepared as described previously (Cho et al., 2010). Human iPSCs were generated from human foreskin fibroblasts (CRL-2097™, ATCC, Manassas, Va.) by ectopic expression of 4 transcription factors such as OCT4, SOX2, KLF4, and c-MYC as previously described (Takahashi et al., 2007). Human iPSC was maintained on MMC-treated mouse embryonic fibroblast feeder layers in Dulbecco's modified Eagle medium (DMEM)/F-12 (Invitrogen) supplemented with 20% Knockout Serum Replacement (Invitrogen), 1% non-essential amino acids (Invitrogen), 1% penicillin—streptomycin (Invitrogen), 0.1 mM b-mercaptoethanol (Sigma-Aldrich), and 4 ng/ml basic fibroblast growth factor (bFGF; R&D Systems). The medium was changed daily.

### Example 1.2

### Generation of Mouse ESCs Expressing tdTomato Fluorescence

Lentiviruses were generated by transfecting FUtdTW (Addgene plasmid 22478) (Rompani and Cepko, 2008) with pMD2.G (Addgene plasmid 12259), pMDLg/pRRE (Addgene plasmid 12251) and pRSV-Rev (Addgene plasmid 12253) (Dull et al., 1998) in 293T cells using jetPEI (Polypus-transfection, 101-10N). Supernatants were collected 48 h after transfection, filtered through a 0.45 μM filter and concentrated by Lenti-X concentrator (Clontech, 631231). Viral particles were resuspended in ESC medium with 4 mg/ml polybrene. E14tg2a cells were incubated in this medium for 24 hours. Selection of ESCs were performed by FACS sorting.

### Example 1.3

### Induction of Mouse PSC-Derived MPCs, Cardioblasts and Cardiomyocytes

For induction of Flk1⁺ MPCs, ESCs and iPSCs were cultured without LIF and plated on a 0.1% gelatin-coated dish at cell density 1-1.5×10³ cells/cm² in the differentiation medium (alpha MEM, Invitrogen) with 10% fetal bovine serum (FBS) (Welgene), 2-mercaptoethanol (Invitrogen), L-glutamine (Invitrogen), and antibiotics (Invitrogen), which was changed every 2 days, for 4.5 days. At day 4.5, differentiated ESCs and iPSCs were harvested with 0.25% trypsin-EDTA and antigen recovery was performed in the differentiation medium for 30 min in an incubator. Then, cells were washed using phosphate buffered saline (PBS)/2% FBS and incubated with biotin-conjugated anti-mouse Flk1⁺ antibody (clone AVAS 12a1, eBioscience) and anti-streptavidin MicroBeads (Miltenyi Biotec). Flk1⁺ MPCs were sorted by AutoMACS Pro Separator (Miltenyi Biotec). For induction of cardioblasts and cardiomyocytes, sorted Flk1⁺ MPCs were plated onto the MMC (AG scientific) treated confluent OP9 cells at a density of 5-10×10³ cells/cm². For the induction of cardiomyocytes in a feeder free system, ESCs were plated on a 0.1% gelatin coated dish at cell density 3×10³ cells/cm² without OP9 cells. Cells were cultured in the differentiation medium which was changed every 2 days.

### Example 1.4

### Induction of Human iPSC-Derived Cardiomyocytes

Human iPSC-derived cardiomyocyte differentiation was induced as previously reported (Uosaki et al., 2011). For cardiomyocyte differentiation, human iPSCs were plated onto Matrigel-coated dishes at a density of 10.0×10⁴ cells/cm² and cultured in mTeSR1 supplemented with 4 ng/mL bFGF for 2-3 days. Then, they were cultured in RPMI+B27 medium (RPMI1640, 2 mM L-glutamine, and 1×B27 supplement without insulin) supplemented with 100 ng/mL of activin A (R&D Systems) for 24 hr and further cultured in the same medium containing 25 ng/mL human BMP4 (R&D Systems) and 50 ng/mL bFGF for 4 days. The culture medium was subsequently changed with RPMI+B27 supplemented with 100 ng/mL Dickkopf-related protein 1 (Dkk1, R&D Systems) and 25 ng/mL vascular endothelial growth factor (VEGF, R&D Systems) for 2 days. At day 7, the culture medium was replaced to RPMI+B27 without growth factors. The medium was changed every 1-2 days. Beating cardiomyocytes were observed at day 8-9.

### Example 1.5

### Reagents

CsA (a gift from Novartis Pharma, Korea), 6-Hydroxy-2,5,7,8-tetramethylchroman-2-carboxylic acid (Trolox, Sigma Aldrich), and EW7197 (Son et al., 2014) were dissolved in dimethyl sulfoxide (DMSO, Sigma Aldrich). Y27632 (Calbiochem) was dissolved in distilled water. Reagents were treated at the time of medium change. DMSO was treated as a control vehicle.

### Example 1.6

### Flow Cytometry Analysis and Cell Sorting

Differentiating Flk1⁺ MPCs on OP9 cells were harvested with 0.25% trypsin-EDTA or dissociation buffer (Invitrogen). To analyze live cells, antigen recovery was performed in the differentiation medium for 30 min in an incubator and the cells were incubated for 20 min with the following antibodies: phycoerythrin (PE)-conjugated anti-mouse PDGFRα (clone APA5, eBioscience), allophycocyanin (APC)-conjugated anti-mouse Flk1 (clone AVAS 12a1, BioLegend), APC-conjugated anti-mouse PDGFRβ (clone APBS, eBioscience), PE-conjugated anti-mouse CD31 (clone 390, eBioscience), APC-conjugated anti-mouse CD144 (clone BV13, eBioscience), PE-conjugated anti-mouse CD41 (clone MWReg30, BD Pharmingen™), and APC-conjugated anti-mouse CD45 (clone 30-F11, eBioscience) antibodies. To analyze cTnT⁺ cells, the cells were permeabilized using Cytofix/Cytoperm solution (BD Biosciences) for 15 min. After permeabilization, the cells were incubated for 30 min with anti-mouse cTnT (Clone 13-11, Thermo Scientific) monoclonal antibody. After washing in 10% Perm/Wash buffer (BD Biosciences), the cells were incubated for 10 min with Cy5-conjugated anti-mouse IgG antibody (Invitrogen). The cells were washed with 10% Perm/Wash buffer (BD Biosciences) and then analyzed. In live cell analysis and sorting, dead cells were excluded using 4,6-diamidino-2-phenylindole (DAPI, Invitrogen) and OP9 cells were excluded from Flk1⁺ MPCs by gating in flow cytometry. The differentiated cardiomyocytes were sorted using αMHC-GFP. Analyses and sorting were performed by FACS Aria II (Beckton Dickinson). Data were analyzed using FlowJo Version 7.5.4 software (TreeStar).

### Example 1.7

### Immunofluorescence Staining and Visualization of Cells

The cells were fixed with 2% paraformaldehyde (PFA) and blocked with 5% goat (or donkey) serum in PBST (0.1% Tween 20 in PBS) for 1 hr at room temperature (RT). The cells were stored overnight at 4° C. with the following primary antibodies: anti-mouse cTnT (Clone 13-11, Thermo Scientific) and anti-mouse α-actinin (clone EA-53, Sigma Aldrich) monoclonal antibody, anti-mouse PDGFRα (clone APA5, eBioscience), anti-rabbit Flk1 (clone D5B1, Cell Signaling), and anti-rabbit Nkx 2.5 (Santa Cruz Biotechnology) polyclonal antibodies. After being washed with PBST 3 times, the cells were incubated for 2 hr at room temperature with the following secondary antibodies: Cy3-conjugated anti-mouse IgG antibody (Invitrogen), FITC-conjugated anti-rat IgG (Jackson ImmunoResearch), and Cy3-conjugated anti-rabbit IgG (Jackson ImmunoResearch) antibodies. After being stained with the antibodies, the cells were mounted in fluorescent mounting medium (DAKO). Nuclei were stained with 4,6-diamidino-2-phenylindole (DAPI, Invitrogen). Immunofluorescence staining of mitochondria was performed using MitoTracker Orange CMTMRos probe (Invitrogen) and cells were incubated with probe for 30 to 60 min at 37° C. in serum free medium before fixation. Immunocytochemistry stained images were obtained using an LSM510 confocal fluorescence microscope (Carl Zeiss). Live images of cardiomyocyte differentiation process and αMHC GFP⁺ cardiomyocytes were obtained using Axiovert 200M microscope (Carl Zeiss) equipped with AxioCam MRm (Carl Zeiss). Images were analyzed using Image J software (http://imagej.nih.gov/ij/, 1.47V, NIH, USA). Phase-contrast images including beating cardiomyocytes were obtained using an Infinity X digital camera and DpxView LE software (DeltaPix).

### Example 1.8

### Quantitative Real Time PCR

Total RNA was extracted using Trizol RNA extraction kit (Invitrogen) according to the manufacturer's instructions. Total RNA was reverse transcribed into cDNA using GoScript™ cDNA synthesis system (Promega). cDNA was applied for quantitative real-time PCR using FastStart SYBR Green Master mix (Roche) and Bio-rad S1000 Thermocycler with the indicated primers (Table 1). Beta-actin was used as a reference gene and the results were presented as relative expression to control using the ΔΔCt method.

TABLE 1
Primers for real time PCR

| | | |
|---|---|---|
| Mouse | Forward | 5′-TCTTTCCACCAGGCCCCCGGC |
| oct3/4 | | TC-3′ (SEQ ID NO: 1) |
| | Reverse | 5′-TGCGGGCGGACATGGGGAGAT |
| | | CC-3′ (SEQ ID NO: 2) |
| Mouse | Forward | 5′-AGGGTCTGCTACTGAGATGCT |
| nanog | | CTG-3′ (SEQ ID NO: 3) |
| | Reverse | 5′-CAACCACTGGTTTTTCTGCCA |
| | | CCG-3′ (SEQ ID NO: 4) |
| Mouse | Forward | 5′-TAGAGCTAGACTCCGGGCGAT |
| sox2 | | GA-3′ (SEQ ID NO: 5) |
| | Reverse | 5′-TTGCCTTAAACAAGACCACGA |
| | | AA-3′ (SEQ ID NO: 6) |
| Mouse | Forward | 5′-CCATCGTTCCTGTACGCAGAA |
| mesp1 | | ACAG-3′ (SEQ ID NO: 7) |
| | Reverse | 5′-AGACAGGGTGACAATCATCCG |
| | | TTGC-3′ (SEQ ID NO: 8) |
| Mouse | Forward | 5′-CTATGCTCATCGGAACAGCTC |
| brachyury | | TCCA-3′ (SEQ ID NO: 9) |
| | Reverse | 5′-CTCACAGACCAGAGACTGGGA |
| | | TAC-3′ (SEQ ID NO: 10) |
| Mouse | Forward | 5′-CCCTCTTTGTCATTCTTCGCT |
| gata4 | | GGAG-3′ (SEQ ID NO: 11) |
| | Reverse | 5′-GATTTGCGGTTGCTCCAGAAA |
| | | TCGTG-3′ (SEQ ID NO: 12) |
| Mouse | Forward | 5′-CACGCCTTTCTCAGTCAAAGA |
| nkx2.5 | | CATCC-3′ (SEQ ID NO: 13) |
| | Reverse | 5′-CTGGGAAAGCAGGAGAGCACT |
| | | TGG-3′ (SEQ ID NO: 14) |
| Mouse | Forward | 5′-CGCCTCTGGAGCCTGATTCCA |
| tbx5 | | AAG-3′ (SEQ ID NO: 15) |
| | Reverse | 5′-GTGCCCACTTCGTGGAACTTC |
| | | AGC-3′ (SEQ ID NO: 16) |
| Mouse | Forward | 5′-AGACCCTCTCAGTCCCTTGCA |
| isl1 | | TC-3′ (SEQ ID NO: 17) |
| | Reverse | 5′-CATCTCCACTAGTTGCTCCTT |
| | | CATG-3′ (SEQ ID NO: 18) |
| Mouse | Forward | 5′-CAAGATCAAGACACTGCGCCT |
| hand2 | | GG-3′ (SEQ ID NO: 19) |
| | Reverse | 5′-TCGTTGCTGCTCACTGTGCTT |
| | | TTC-3′ (SEQ ID NO: 20) |
| Mouse | Forward | 5′-GACCTGTGTGCAGTCCCTGTT |
| tnnt2 | | CAG-3′ (SEQ ID NO: 21) |
| | Reverse | 5′-CTTGCTCGTCCTCCTCTTCTT |
| | | CAC-3′ (SEQ ID NO: 22) |
| Mouse | Forward | 5′-ATCAGACCTGAAGGAGACCTA |
| myl7 | | TTCC-3′ (SEQ ID NO: 23) |
| | Reverse | 5′-AAGGCACTCAGGATGGCTTCC |
| | | TC-3′ (SEQ ID NO: 24) |
| Mouse | Forward | 5′-GCGCCGTGTGATTTACGT |
| pgc1α | | T-3′ (SEQ ID NO: 25) |
| | Reverse | 5′-AAAACTTCAAAGCGGTCTCTC |
| | | AA-3′ (SEQ ID NO: 26) |
| Mouse | Forward | 5′-GTGTCTGTGCCCACACTCCTG |
| connexin43 | | TAC-3′ (SEQ ID NO: 27) |
| | Reverse | 5′-CTCAGCAGGCCACCTCTCATC |
| | | TTC-3′ (SEQ ID NO: 28) |
| Mouse | Forward | 5′-GCTCTTTTCCAGCCTTCCT |
| beta actin | | T-3′ (SEQ ID NO: 29) |
| | Reverse | 5′-CTTCTGCATCCTGTCAGCA |
| | | A-3′ (SEQ ID NO: 30) |

### Example 1.9

### Microarray Analysis

For control and test RNAs, synthesis of target cRNA probes and hybridization were performed using Agilent's Low RNA Input Linear Amplification kit (Agilent Technology, USA) according to the manufacturer's instructions. Briefly, each 1 μg total RNA and T7 promoter primer mix were incubated at 65° C. for 10 min. cDNA master mix (5× First strand buffer, 0.1M DTT, 10 mM dNTP mix, RNase-Out, and MMLV-RT) was prepared and added to the reaction mixer. The samples were incubated at 40° C. for 2 hr and then the RT and dsDNA synthesis was terminated by incubating at 65° C. for 15 min. The transcription master mix was prepared as the manufacturer's protocol (4× Transcription buffer, 0.1M DTT, NTP mix, 50% PEG, RNase-Out, Inorganic pyrophosphatase, T7-RNA polymerase, and Cyanine 3-CTP). Transcription of dsDNA was preformed by adding the transcription master mix to the dsDNA reaction samples and incubating at 40° C. for 2 hr. Amplified and labeled cRNA was purified on cRNA Cleanup Module (Agilent Technology) according to the manufacturer's protocol. Labeled cRNA target was quantified using ND-1000 spectrophotometer (NanoDrop Technologies, Inc., Wilmington, Del.). After checking labeling efficiency, fragmentation of cRNA was performed by adding 10× blocking agent and 25× fragmentation buffer and incubating at 60° C. for 30 min. The fragmented cRNA was resuspended with 2× hybridization buffer and directly pipetted onto assembled Agilent's Mouse Oligo Microarray (44K). The arrays hybridized at 65° C. for 17 hr using Agilent Hybridization oven (Agilent Technology, USA). The hybridized microarrays were washed according to the manufacturer's washing protocol (Agilent Technology, USA). The hybridized images were scanned using Agilent's DNA microarray scanner and quantified with Feature Extraction Software (Agilent Technology, Palo Alto, Calif.). All data normalization and selection of fold-changed genes were performed using GeneSpringGX 7.3 (Agilent Technology, USA). The averages of normalized ratios were calculated by dividing the average of normalized signal channel intensity by the average of normalized control channel intensity. Functional annotation of genes was performed according to Gene Ontology™ Consortium (geneontology.org/index.shtml) by GeneSpringGX 7.3. Gene classification was based on searches done by BioCarta (biocarta.com/), GenMAPP (genmapp.org/), DAVID (david.abcc.ncifcrf.gov/), and Medline databases (ncbi.nlm.nih.gov/).

### Example 1.10

### Assays for Cell Cycle

To determine the cell cycle, BrdU/7-AAD cell cycle analysis was performed according to the manufacturer's instructions (BD Pharmingen™). Briefly, cells were incubated with BrdU (1 mM) for 1 hr, dissociated with 0.25% trypsin-EDTA. The cells were then fixed, permeabilized, and fixed once more, followed by 1 hr incubation with DNase I (200 U) at 37° C. After incubation with APC-conjugated anti-BrdU antibody for 20 min at RT, the cells were stained with 7-AAD. The cells were analyzed by FACS Aria II and the data were analyzed using FlowJo software.

### Example 1.11

### Transmission Electron Microscopic Analysis

The cells were fixed in 2.5% glutaraldehyde in PBS solution at 4° C. overnight, and then with 1% osmium tetroxide in PBS for 2 hr. The tissues were washed, dehydrated, and embedded, and then semi-thin sections were cut (0.5-1 μm). Further ultra-sectioning (60-90 nm) was performed and then the slices were double stained with uranyl acetate and lead citrate and imaged using a JEM 1200 EX2 electron microscope (Jeol, Japan). Developed images were scanned on a flatbed scanner (Umax PowerLook 1100; Fremont, Calif., USA) and analyzed using Image J software (http://imagej.nih.gov/ij/, 1.47V, NIH, USA).

### Example 1.12

### Scanning Electron Microscopic Analysis

For vascular fixation, harvested heart was fixed in 8% paraformaldehyde for overnight, embedded with paraffin, and sectioned. The samples were freeze-dried in a lyophilizer for 24 h, and mounted on stubs and coated with ion-exchanger (KIC-1A, COXEM) operated at 6 mA for 60 s. Images were acquired using scanning electron microscopy (SEM, S-4800, Hitachi) operated at 15 kV, 7 A.

### Example 1.13

### Electrophysiology

Action potentials (APs) and ion currents were recorded from beating cardiomyocytes placed onto the recording chamber of microscope by using Axopatch 200B amplifier (Axon Instrument) at room temperature (23±1° C.). Normal Tyrode (NT) solutions were perfused during seal formation and it contained 143 mM NaCl, 5.4 mM KCl, 0.5 mM MgCl₂, 1.8 mM CaCl₂, 5.5 mM glucose, and 5 mM N-[2-hydroxyethyl]piperazine-N-[2-ethanesulfonic acid] (HEPES). pH was adjusted to 7.4 with 1 M NaOH. For measurement of APs or K⁺ current, we used K⁺-rich pipette filling solutions containing 140 mM KCl, 1 mM MgCl₂, 5 mM MgATP, 5 mM ethyleneglycol-bis(β-aminoethyl ether)-N,N,N′,N′-tetraacetic acid (EGTA), 5 mM glucose, and 5 mM HEPES, titrated to pH 7.2 with 1 mol/L KOH. For measurement of Ca²⁺ or Na⁺ currents, the bathing solution was switched from NT to a solution containing 120 mM NaCl, 5 mM KCl, 1 mM MgCl₂, 3.6 mM CaCl₂, 20 mM TEA, and 10 mM HEPES, titrated to pH 7.4 with 1 M NaOH. Pipette filling solutions contained 140 mM CsCl, 5 mM glucose, 3 mM MgATP, 10 mM EGTA, and 10 mM HEPES, titrated to pH 7.2 with 1 M CsOH. Patch pipettes were pulled from thin-walled borosilicate capillaries (Clark Electromedical Instruments) using a PP-83 vertical puller (Narishige). GΩ seal formation and membrane rupture were achieved by applying negative pressure onto the membrane patch and only whole-cell patches with series resistance <5 MS2 were selected for recording. All the recordings were carried out at least 5 minutes after achieving whole-cell configuration to allow cells being completely dialyzed with pipette-filling solution. Spontaneous APs were recorded in current-clamp mode while ion currents were recorded in voltage-clamp mode. The voltage and current signals were filtered at 10 kHz, 4-pole Bessel type low-pass filter and sampled at a rate of 4 kHz for voltage and 27 kHz for ion current. All experimental parameters, such as pulse generation and data acquisition, were controlled using our own software (PatchPro). The liquid junction potentials between bathing and pipette filling solution, which were calculated based on ionic mobility, were <5 mV. TEA, TTX, and mibefradil were used to block delayed rectifying K⁺ channels, voltage-gated Na⁺ channels, and T-type Ca²⁺ channels, respectively.

### Example 1.14

### Western Blotting

Cells were homogenized in lysis buffer (20 mM Tris-HCl, 150 mM NaCl, 10 mM EDTA, 50 mM NaF, and 25 mM NaVO₄) and centrifuged at 13,000 rpm for 10 min at 4° C. After centrifugation, 30 μg of proteins were subjected to 10% sodium dodecyl sulfate-polyacrylamide gels and transferred onto nitrocellulose membranes. Membranes were incubated with Cav3.2 (Alomone), Kir2.1 (Alomone), Nav1.3 (Alomone) and α-tubulin primary antibody (Santa cruz). Secondary antibodies were subjected with a goat anti-rabbit or mouse IgG (Abcam). The immunoreactive protein bands were detected using SuperSignal West Pico enhanced chemiluminescence system and visualized using LAS-3000 PLUS (Fuji Photo Film Company, Kanagawa, Japan).

### Example 1.15

### Preparation of Acute MI Model in Mouse and Cell Transplantation

Animal care and experimental procedures were performed under the approval (KA2013-40) of the Animal Care Committee of KAIST. MI was induced by ligation of the left anterior descending coronary artery in 9 weeks aged BALB/c nude male mice for avoiding immune reaction. The heart was exposed through a left thoracotomy, and the middle portion of left anterior descending coronary artery was permanently ligated by 8-0 prolene thread. Infarction of the anterior wall of the left ventricle was confirmed in each mouse by the presence of a pale anterior wall color and myocardial hypokinesis after coronary artery ligation. Immediately after coronary artery ligation, 100 μL culture medium containing 1×10⁶ PCBs and M⁺CMs were intramyocardially injected with a 31-gauge (0.25 mm) insulin syringe into the 3 sites which are border zone surrounding the infarcted area.

### Example 1.16

### Histological Analyses

Before sacrifice, mice were anesthetized with mixtures of ketamine (80 mg/kg) and xylazine (10 mg/kg). For H&E staining, samples were fixed overnight in 4% PFA and embedded in paraffin after tissue processing. For immunofluorescence staining, samples were fixed in 4% PFA, dehydrated in 20% sucrose solution overnight, and embedded in tissue freezing medium (Leica). Samples were blocked with 5% goat (or donkey) serum in 0.01% Trition X-100 in PBS and then incubated for overnight at 4° C. with the following primary antibodies: anti-mouse α-actinin (clone EA-53, Sigma Aldrich) monoclonal antibody, anti-mouse PDGFRα (clone APA5, eBioscience), anti-rat GFP (Millipore), and anti-rabbit Connexin 43 (Invitrogen) polyclonal antibodies. After several washes, the samples were incubated for 2 hr at room temperature (RT) with the following secondary antibodies: Cy3 or FITC-conjugated anti-mouse IgG (Jackson ImmunoResearch), FITC-conjugated anti-rat IgG (Jackson ImmunoResearch), and Cy5-conjugated anti-rabbit IgG (Jackson ImmunoResearch) antibodies. Then the samples were mounted with fluorescent mounting medium (DAKO) and immunofluorescent images were acquired using a Zeiss LSM780 confocal microscope (Carl Zeiss).

### Example 1.17

### Cardiac MRI Analysis

Throughout the experiments, mice were anesthetized with isoflurane delivered by nose cone and their respiratory rate, electrocardiogram, and rectal temperature were monitored. The CINE images were acquired on a 9.4-T high field small animal imaging system (Agilent inc., Palo Alto, Calif., USA) with a 30 mm-diameter millipede volume coil. A stack of short-axis slices covering the heart from the apex to the base was acquired with an ECG triggered and respiratory-gated FLASH sequence with the following parameters: TR/TE=240/2.1 ms; field of view (FOV)=25×25 mm; matrix size=192×192; slice thickness of 0.8 mm; 50 frames per R-R interval; CINE TR=4.76 ms; flip angle at 30°; 16 averages; total scan time=12 m 17 s. During CINE MRI scans, heart rate for the external trigger was 250 ppm.

### Example 1.18

### Transthoracic Echocardiographic Analysis

Transthoracic echocardiographic studies were performed (VIVID 7 dimension system, General Electric-Vingmed Ultrasound, Horton Norway) 14 days after myocardial infarction surgery and cell transplantation under anesthesia. Images were obtained using an i13L transducer (5.3-14.0 MHz, GE Healthcare) with high temporal and spatial resolution. 2-dimensionally targeted M-mode parameters were measured at a level of papillary muscle was visualized in parasternal short axis view during over ≧6 consecutive cardiac beats. All measurements were performed in a blind fashion according to the American Society for Echocardiography.

### Example 1.19

### Statistical Analysis

Values presented are means±standard deviation (SD). The assumption of normality was evaluated using Shaprio-Wilk test. Significant differences between means were determined by unpaired Student t-test or analysis of variance with one-way ANOVA followed by the Student-Newman-Keuls test. The Mann-Whitney test and Krusakl-Wallis ANOVA were performed when data were not normally distributed. Statistical significance was set at p<0.05 or 0.01.

### Example 2

### Results

### Example 2.1

### CsAYTE Promotes PSC Differentiation into Cardiomyocytes

Our previous study (Cho et al., 2014) showed that CsA treatment of PSCs leads to their differentiation into functional cardiomyocytes by altering mitochondrial oxidative metabolism mediated through mPTP inhibition. Under this condition, addition of antioxidants augmented the cardiomyogenic effects of CsA (Cho et al., 2014). Since the inhibition of ROCK or ALK5 signaling also contributes to cardiomyogenesis (Cai et al., 2012; Kitamura et al., 2007; Ryan et al., 2013; Willems et al., 2012), we hypothesized that a combinatorial treatment of PSCs with all four specific reagents may synergistically promote their differentiation into cardiomyocytes.

For the monitoring and tracing of cardiomyocyte differentiation, we not only used the EMG7 embryonic stem cell (ESC) line, which has a transgene of cardiac specific α myosin heavy chain (αMHC) promoter-driven enhanced green fluorescent protein (GFP), but also identified cardiac specific markers, cardiac troponin T (cTnT) and α-actinin in the differentiating PSCs. At day 4.5 after mesodermal induction without leukemia inhibitory factor (LIF) in ESCs, Flk1⁺ MPCs were sorted and plated onto a mitomycin-c (MMC)-treated OP9 feeder cell layer, and four specific reagents were added to the differentiation medium.

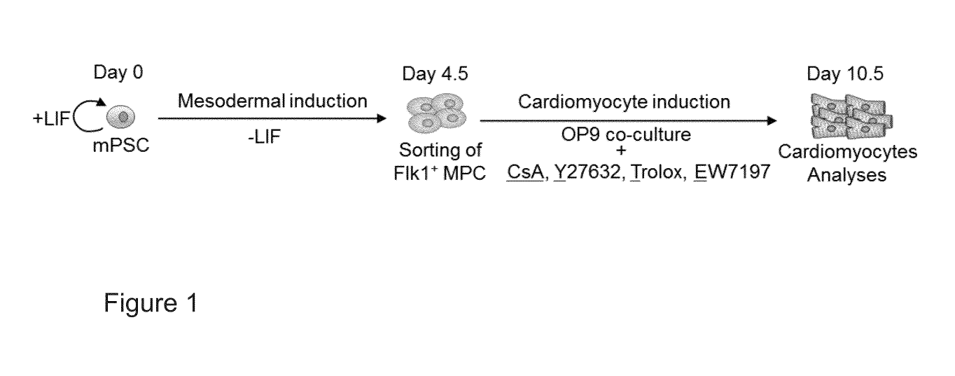

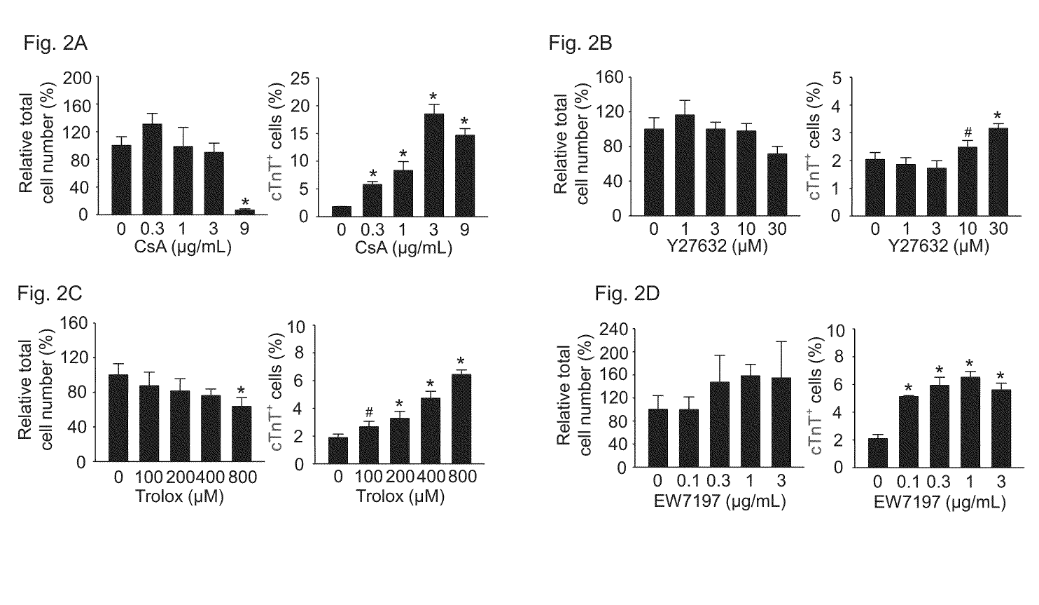

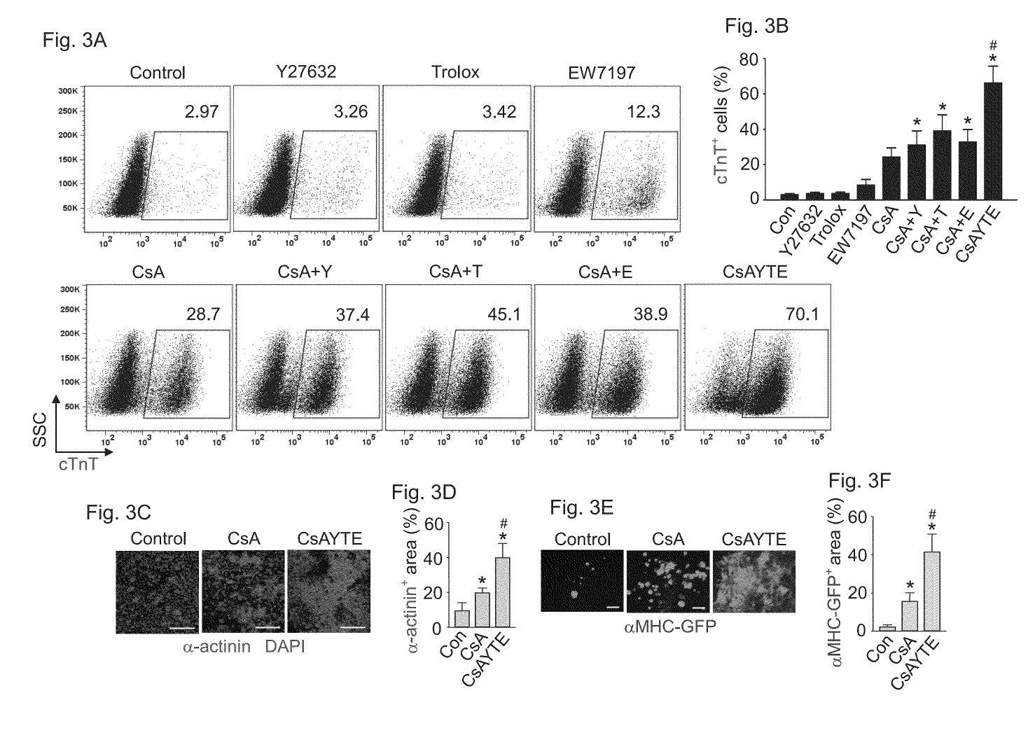

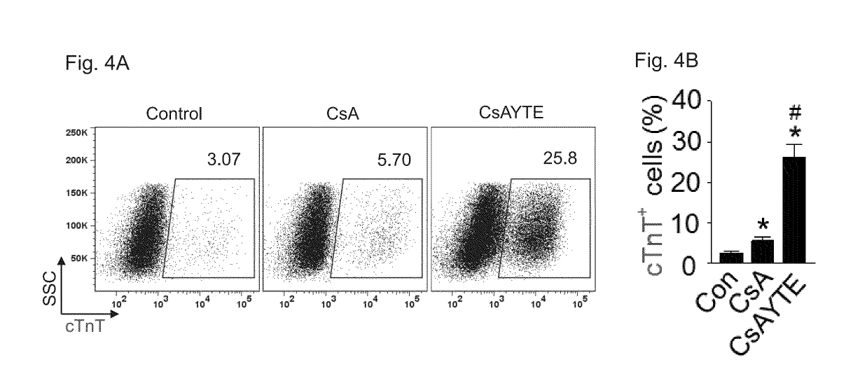

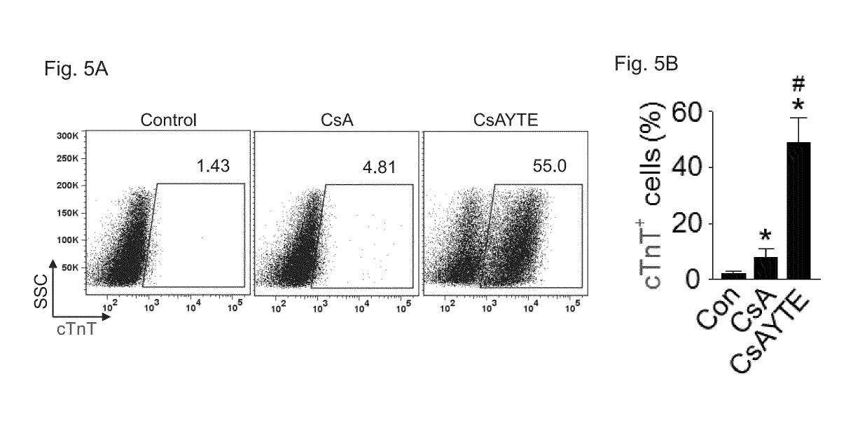

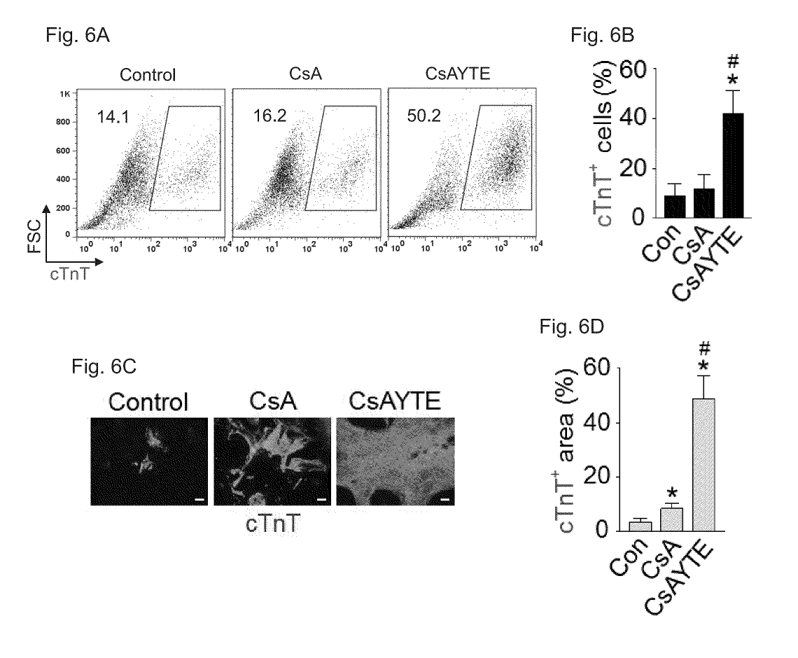

We treated the reagents including, CsA for mPTP inhibition, Y27632 for ROCK inhibition, Trolox as antioxidant, and EW7197 for ALK5 inhibition. The effect of reagents on differentiation into cardiomyocyte was analyzed at day 10.5 (FIG. 1). Dose optimization was determined by total and relative number of differentiated cTnT⁺ cells; optimal dose of each reagent was 3 μg/mL of CsA, 10 μM of Y27362, 400 μM of Trolox, and 1 μg/mL of EW7197 (FIGS. 2A-D). Optimal dose of each reagent induced Flk1⁺ MPC differentiation into cTnT⁺ cells on average from 3.78% to 24.5%, and the combination of each reagent with CsA further promotes differentiation on average from 31.3% to 39.3% (FIGS. 3A and 3B). However, the combination of all four reagents, CsAYTE strikingly promoted Flk1⁺ MPC differentiation into cTnT⁺ cells to ˜70% compared to control vehicle (21.1-fold), to each reagent (2.7- to 17.5-fold), and to each reagent in combination with CsA (1.7- to 2.1-fold) (FIGS. 3A and 3B). Accordingly, CsAYTE profoundly increased 1) area of self-beating cells, 2) area of α-actinin⁺ cells up to 40.0% (FIGS. 3C and 3D), and 3) area of αMHC-GFP⁺ cells up to 41.5% (FIGS. 3E and 3F). Moreover, in a feeder-free culture condition, CsAYTE promoted differentiation into cTnT⁺ cells to 25.8% compared to 3.1% and 5.7% in control vehicle and CsA alone (FIGS. 4A and 4B). Similarly, CsAYTE also increased the differentiation of mouse iPSC-derived Flk1⁺ MPCs into cTnT⁺ cells to 55.0% (FIGS. 5A and 5B). Furthermore, CsAYTE also enhanced the number of human iPSC-derived cTnT⁺ cells to 50.2% compared to control vehicle (14.1%) and CsA alone (16.2%); area of cTnT⁺ cells increased to 48.8% compared to control vehicle (3.56%) and CsA alone (8.38%). (FIGS. 6A-D). In contrast, combination of CsA with other signaling modulators, such as PI3-kinase inhibitor (LY294002), MEK/ERK inhibitor (PD98059), PKA inhibitor (KT5720), PKC inhibitor (Go6976), PKG inhibitor (KT5823), mTOR inhibitor (rapamycin), GSK3β inhibitor (CHIR99021), notch inhibitor (DAPT), AMPK inhibitor and activator (Compound C and AICAR), MLC kinase inhibitor (ML7), or PPARα inhibitor (GW6471), rather inhibited or did not affect Flk1⁺ MPC differentiation into cTnT⁺ cells (data not shown). Thus, CsAYTE is a strong inducer of cardiomyocyte differentiation from PSCs with significantly higher efficiency compared to recently developed methods (Cao et al., 2012; Cho et al., 2014; Kattman et al., 2011).

### Example 2.2

### CsAYTE Induces Flk1⁺ MPC Differentiation into PDGFRα⁺Flk1⁻ Cardioblasts

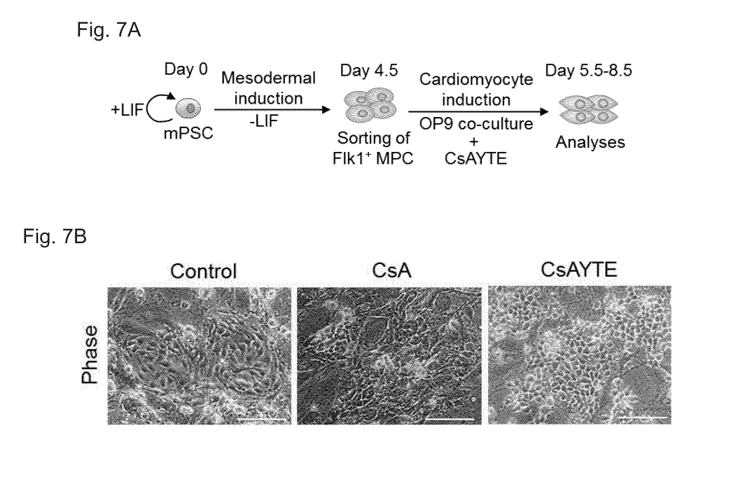

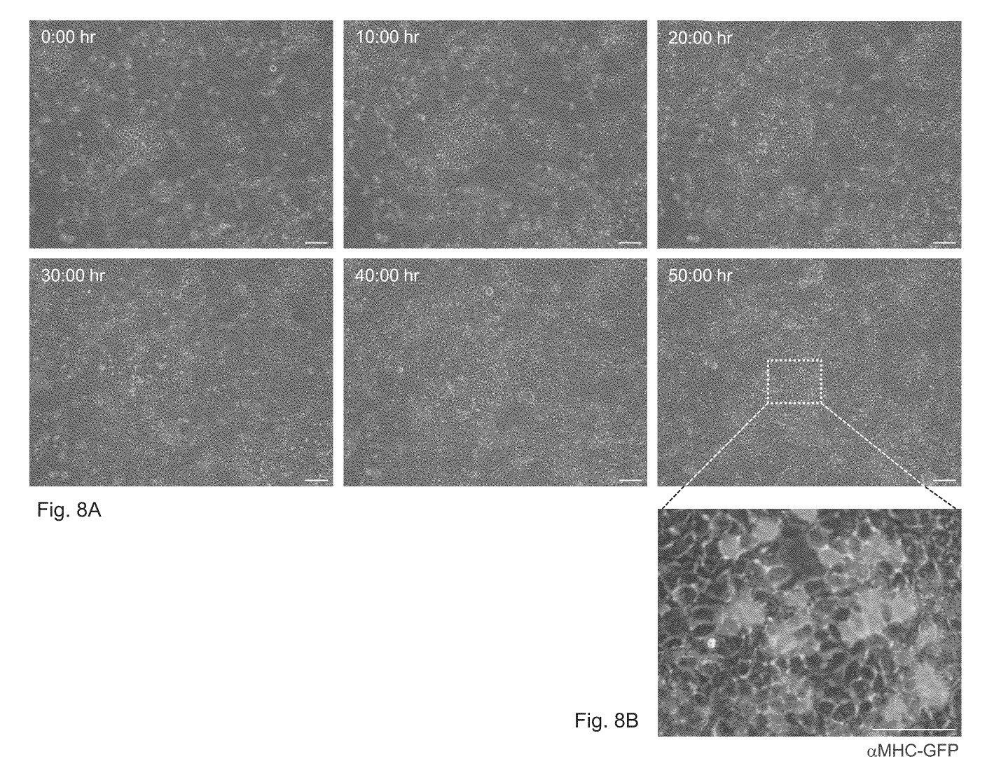

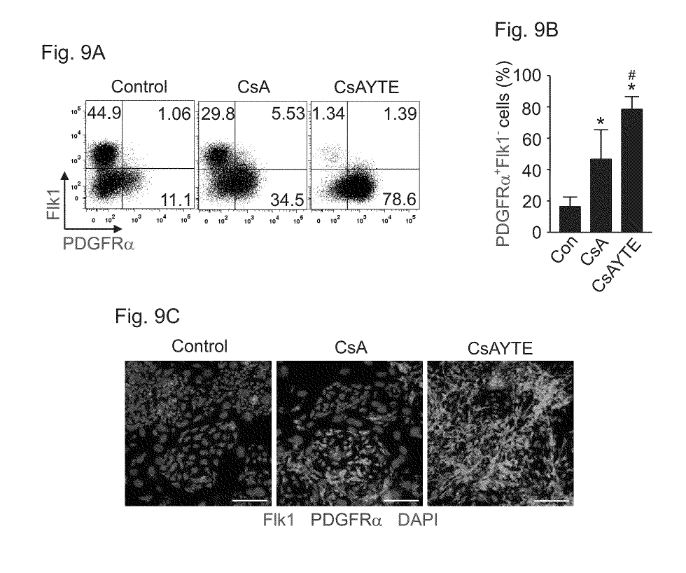

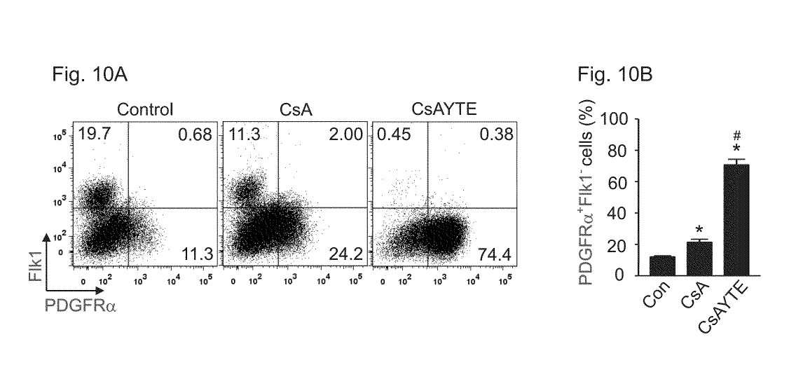

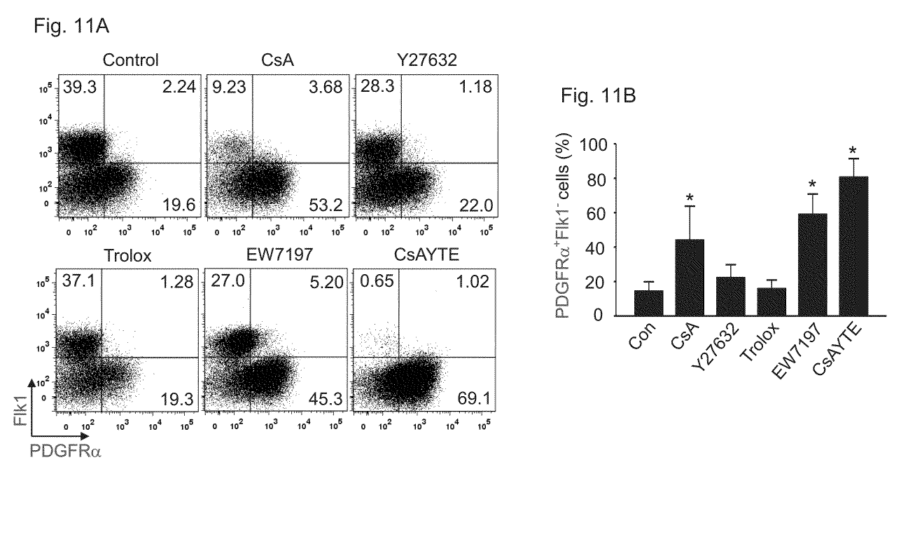

In the process of differentiating Flk1⁺ MPCs into cardiomyocytes (FIG. 7A), the morphology of cells changed homogeneously to small and round shapes within a day after CsAYTE treatment, while it did not exhibit apparent changes with control vehicle or CsA alone (FIG. 7B). These morphologically homogeneous cells rapidly expanded (FIG. 8A), started beating, and expressed αMHC-GFP (FIG. 8B) throughout the course of differentiation. Therefore, this homogeneous cell population exhibits the hallmark features of cardiac precursors, and we define this cell population as “cardioblasts”. To further identify and characterize this cell population based on surface marker expressions, we screened several previously reported cardiovascular progenitor markers, such as Flk1, PDGFRα, PDGFRβ, CXCR4, ALCAM, and c-kit (Bondue et al., 2011; Hirata et al., 2007; Nelson et al., 2008; Scavone et al., 2013; Yamashita et al., 2005; Zaruba et al., 2010). Among them, only PDGFRα was expressed in most of these putative cardioblasts, while the expression of Flk1 was abruptly reduced in these cardioblasts (FIGS. 9A and 9C). Thus, CsAYTE strongly induced Flk1⁺ MPC differentiation into and consequential expansion of PDGFRα⁺ Flk1⁻ cardioblasts (hereafter called as “PCBs”) up to 78.6% and 74.4% from both ESC and iPSC-derived Flk1⁺ MPCs, respectively, within a day, while control vehicle and CsA treatment alone resulted in less than 12% and 35%, respectively. (FIGS. 9A, 9B, 10A and 10B). These findings imply that while the majority of control vehicle and CsA-induced PCBs differentiate into non-cardiomyocyte lineages, most CsAYTE-induced PCBs differentiate into cardiomyocytes. Therefore, we further investigated the PCB differentiation efficiency of each reagent in CsAYTE. Among the four reagents, CsA and EW7197 induced Flk1⁺ MPC differentiation into PCBs up to 53.2 and 45.3%, whereas Y27632 and Trolox induced 22.0% and 19.3%. These data indicate that inhibitions of mPTP and ALK5 are crucial for the induction of Flk1⁺ MPC differentiation into PCBs (FIGS. 11A and 11B).

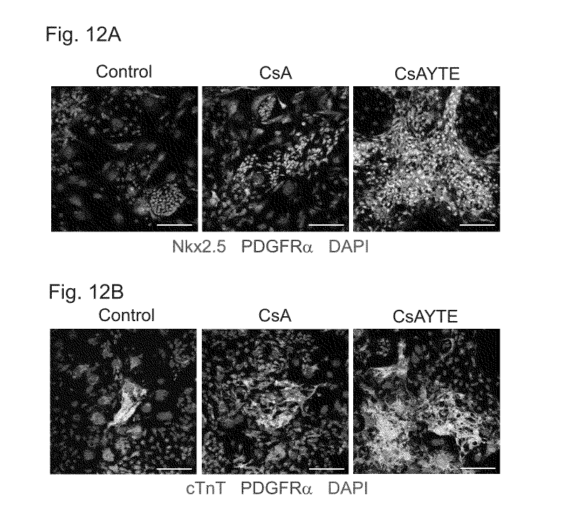

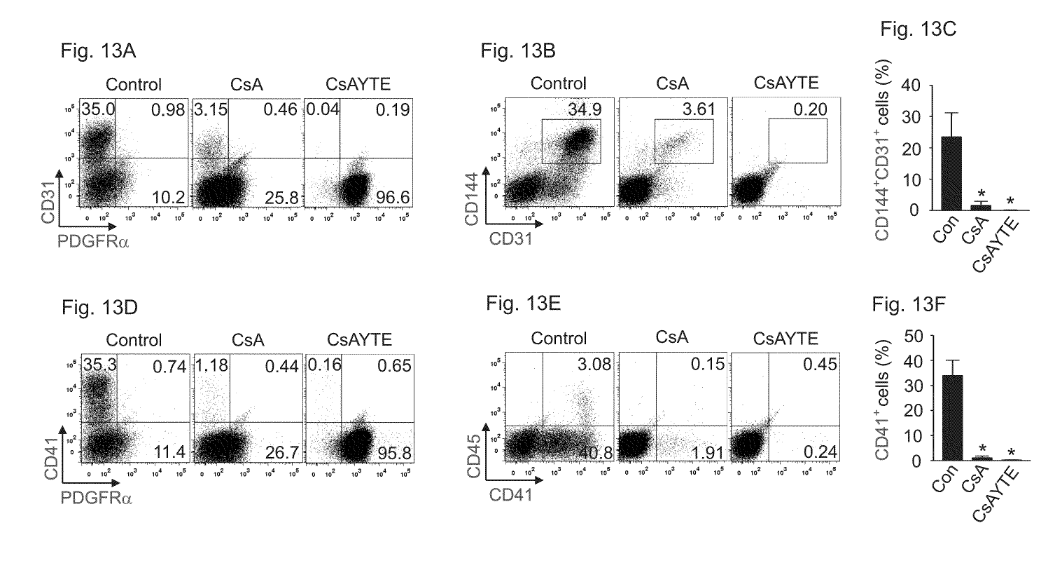

Importantly, expression of PDGFRa was highly co-localized with Nkx2.5, a representative cardiac transcription factor, and cTnT in the PCBs incubated with CsAYTE compared to control vehicle and CsA (FIGS. 12A and 12B). In contrast, PDGFRα expression was not observed in CD31⁺ endothelial cells and CD41⁺ early hematopoietic cells regardless of any reagents treated (FIGS. 13A and 13D). Accordingly, CsAYTE markedly reduced the differentiation into CD144⁺CD31⁺ endothelial cells (FIGS. 13B and 13C) and CD41⁺ early hematopoietic cells to less than 1% (FIGS. 13E and 13F) from Flk1⁺ MPCs. These data indicate that CsAYTE selectively promotes the differentiation of cardiac lineage over endothelial or hematopoietic lineages.

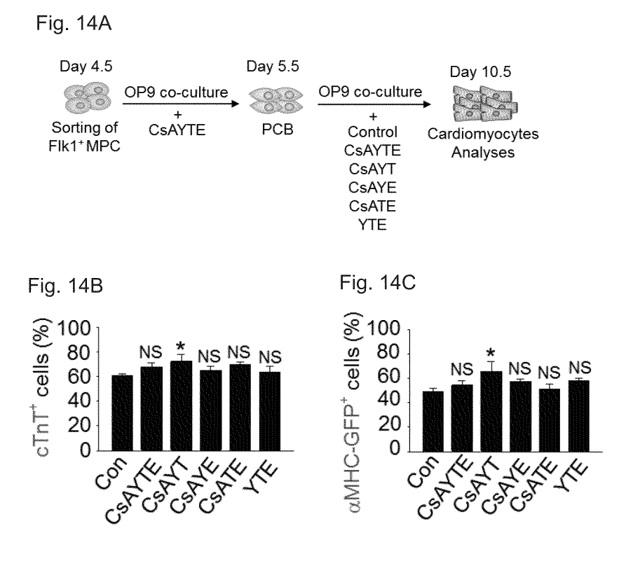

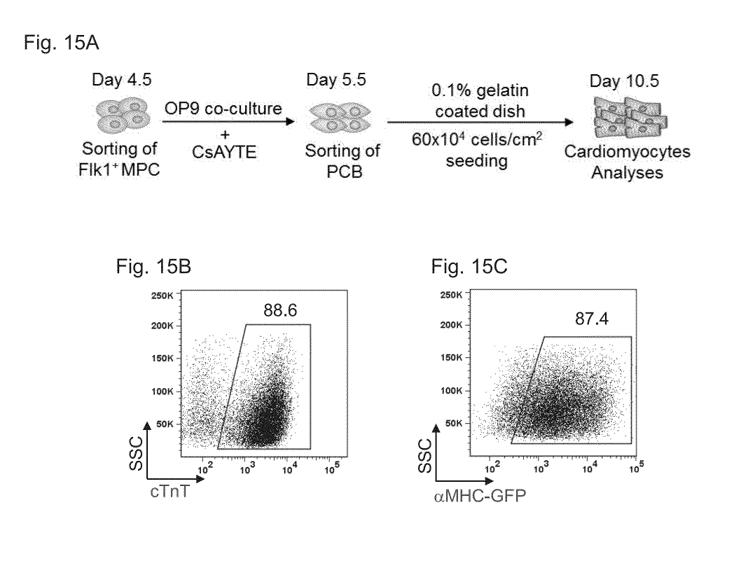

To test whether continuous CsAYTE treatment is required for complete differentiation of PCBs into cardiomyocytes, we removed CsAYTE from the differentiation medium at day 5.5 after PCB induction and monitored the population of cTnT⁺ and αMHC-GFP⁺ cells over time (FIG. 14A). Interestingly, there were no significant differences in the population of cTnT⁺ and αMHC-GFP⁺ cells at day 10.5 between control vehicle and CsAYTE (FIGS. 14B and 14C). Most importantly, sorted PCBs can mostly differentiated into cTnT⁺ and αMHC-GFP⁺ cells in OP9 feeder cell-free condition without CsAYTE treatment (FIGS. 15A-C). These data indicate that PCBs do not require continuous CsAYTE-induced signaling to differentiate into mature cardiomyocytes once they are committed to the cardiac lineage as cardioblasts. Additionally, we tested whether omitting any of the CsAYTE molecules would affect cardiomyocyte differentiation from PCBs (FIG. 14A). Among the four reagents, only the omission of EW7197 slightly increased the population of cTnT⁺ and αMHC-GFP⁺ cells (FIGS. 14B and 14C). These data also suggest that CsAYTE treatment induces cardioblast commitment but does not affect further cardiomyocyte differentiation and maturation.

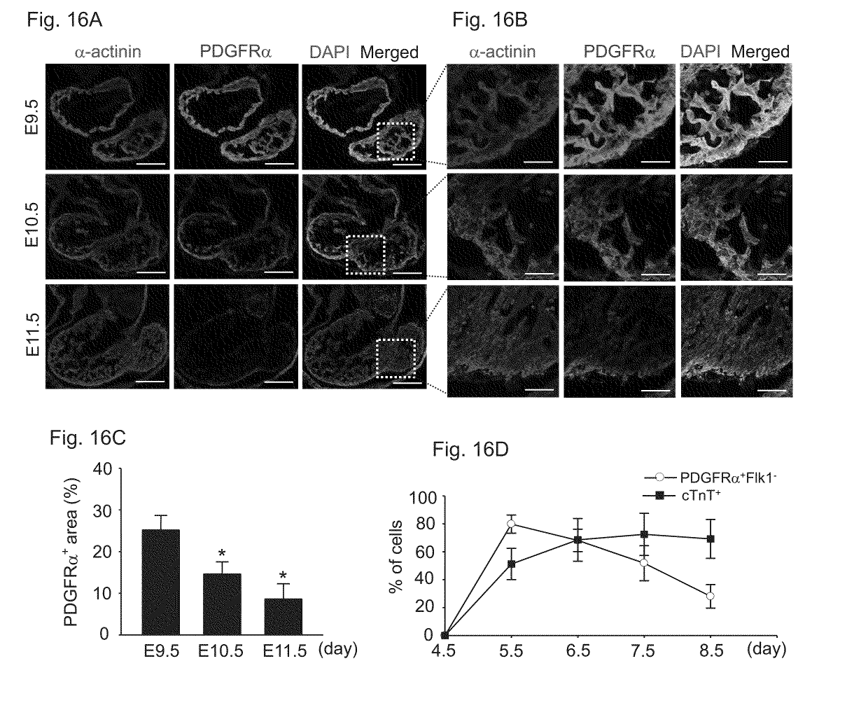

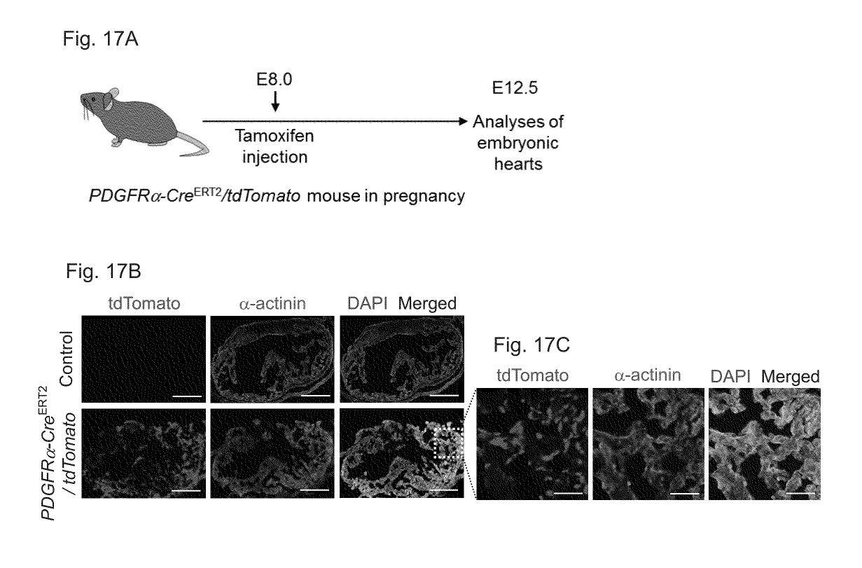

We then wondered whether PDGFRα⁺ cardioblasts exist in the developing heart. Immunofluorescence analyses revealed that PDGFRα was strongly expressed in mouse myocardium and also co-localized with α-actinin expression at E9.5 (FIGS. 16A and 16B). However, PDGFRα expression in myocardium gradually decreased from 25.2% to 8.57% from E9.5 to E11.5 (FIG. 16C). Thus, PDGFRα expression of embryonic myocardium was transiently observed in the mid-embryonic period and its expression pattern was similar to that of PCB differentiation (FIG. 16D). Alternatively, to trace the fate of PDGFRα-expressing cells during embryonic heart development, we used PDGFRα-Cre^{ERT2}/tdTomato mice and their littermates as a control. We injected tamoxifen to the mother at E8.5 and harvested embryos at E12.5 (FIG. 17A). It was noted that the myocardium of PDGFRα-Cre^{ERT2}/tdTomato E12.5 embryo expressed tdTomato and its expression was co-localized with α-actinin⁺ cardiomyocytes (FIGS. 17B and 17C). Thus, these data indicate that PDGFRα⁺ cardioblasts transiently exist in the early embryonic myocardium and can differentiate into cardiomyocytes.

### Example 2.3

### PCBs are Proliferating Cardiac Lineage-Committed Cells in a Morphologically and Functionally Immature State

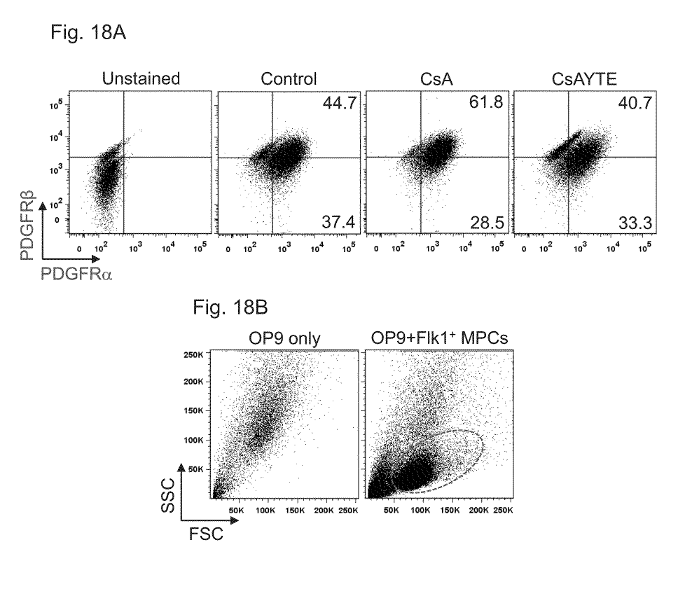

The degree of cardiomyocyte differentiation can be characterized not only by expression patterns of cardiac specific markers including αMHC, cTnT, and α-actinin, but also by functional attributes, such as firing action potentials and global transcriptome analysis (Addis and Epstein, 2013). Therefore, to characterize the novel PCB population, we investigated the properties of PCBs and compared them with PCB-derived differentiated αMHC-GFP⁺ cardiomyocytes (hereafter called as “M⁺CMs”). For this analysis, OP9 feeder cells, which express PDGFRα and β, were excluded from Flk1⁺ MPCs by FACS (FIGS. 18A and 18B).

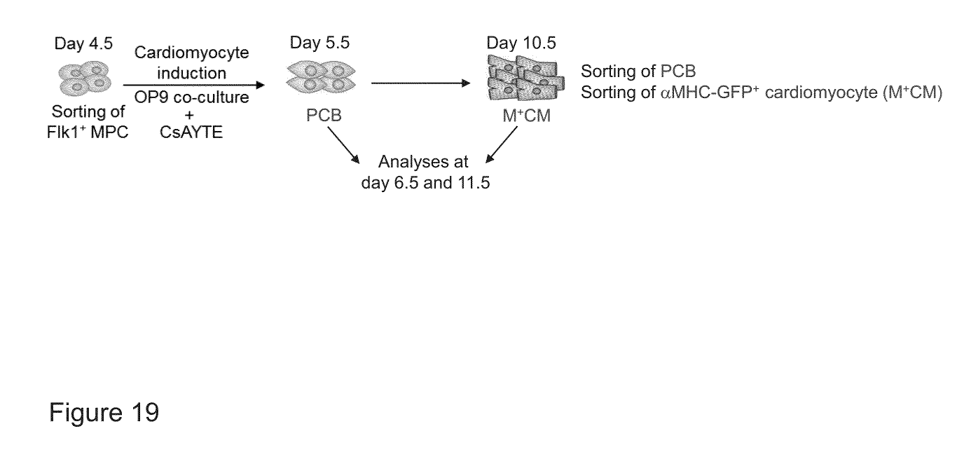

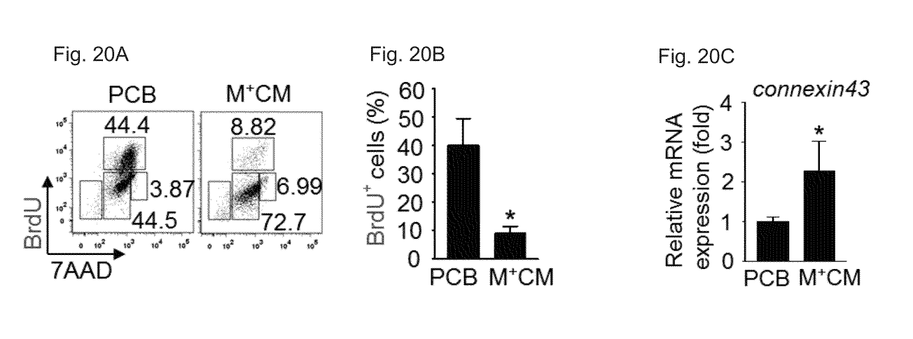

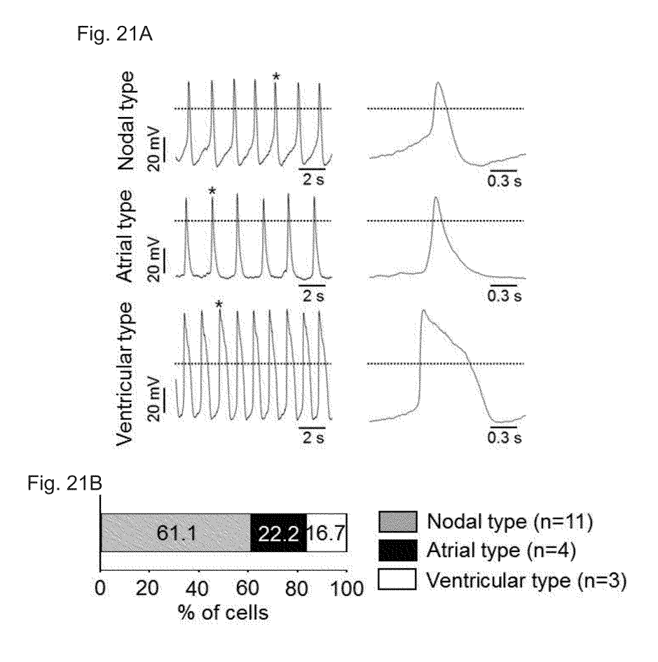

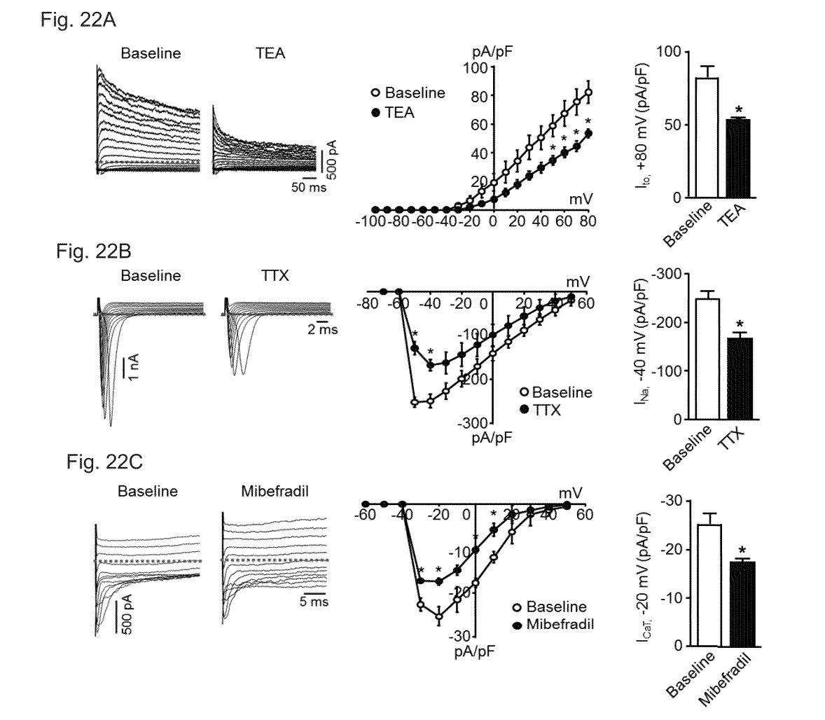

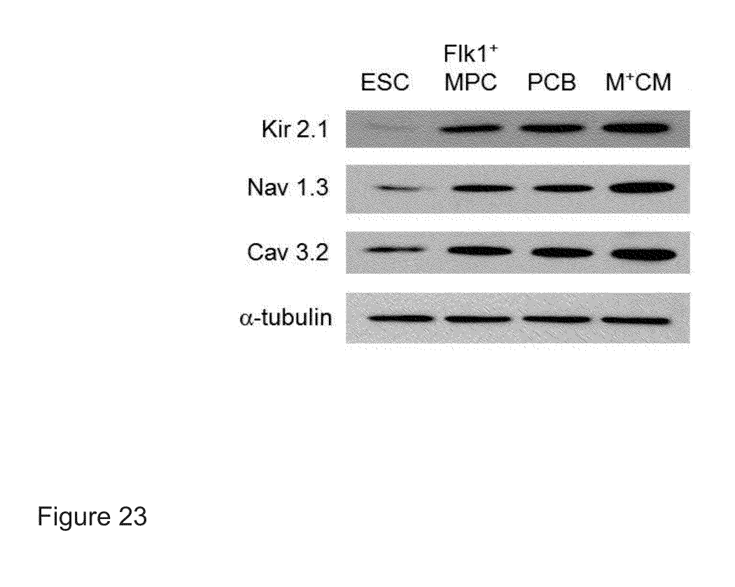

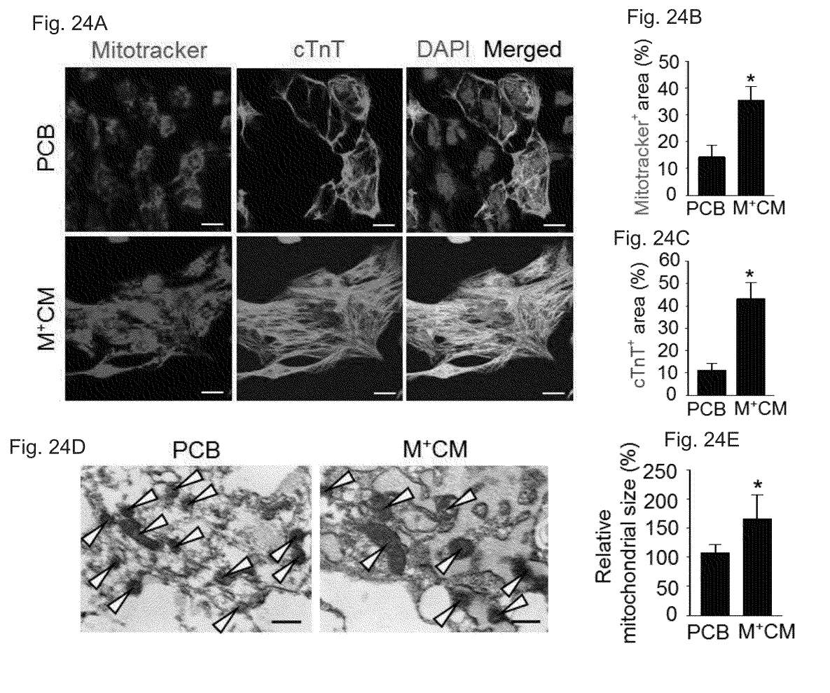

First, to gain insight into the cellular and functional properties between PCBs and M⁺CMs, cells were sorted out at day 5.5 and 10.5, plated onto 0.1% gelatin-coated dishes, analyzed and compared after one day (FIG. 19). As anticipated, the PCB population had a relatively higher (40.1%) proportion of BrdU⁺ proliferating cells than M⁺CMs (9.0%) and mRNA expression levels of connexin43 gap junction were 44% less in PCBs (FIGS. 20A-C). In addition, we noted that PCBs did not show any notable electrical recordings in whole-cell patch clamp analysis, whereas M⁺CMs showed constant and robust firing of spontaneous nodal, atrial, and ventricular action potentials and ion currents, such as delayed rectifier K⁺ current (I_{K}), voltage gated Na⁺ current (I_{Na}), and T-type Ca²⁺ currents (I_{CaT}). (FIGS. 21A, 21B and FIGS. 22A-C). These ion currents were inhibited by ion channel blockades, such as potassium channel blocker, tetraethylammonium (TEA, 20 mM), sodium channel blocker, tetrodotoxin (TTX, 1 μM), and calcium channel blocker, mibefradil (1 μM) (FIGS. 22A-C). These data clearly indicate that M⁺CMs, not PCBs, have electrical properties and function. Consistently, although PCBs expressed ion channels, including Kir 2.1, Nay 1.3, and Cav 3.2, their levels were less than those in M⁺CMs (FIG. 23). These data suggest that the ion channels and contractile structures are not yet properly coupled in the PCBs, while they are well coupled in the M⁺CMs. Furthermore, compared to M⁺CMs, Mitotracker⁺ mitochondria and cTnT⁺ sarcomere area were 21% and 32% less, respectively (FIGS. 24A-C). Transmission electron microscope images also showed less developed mitochondrial cristae and smaller mitochondrial size (white arrow heads) in PCBs (FIGS. 24D and 24E).

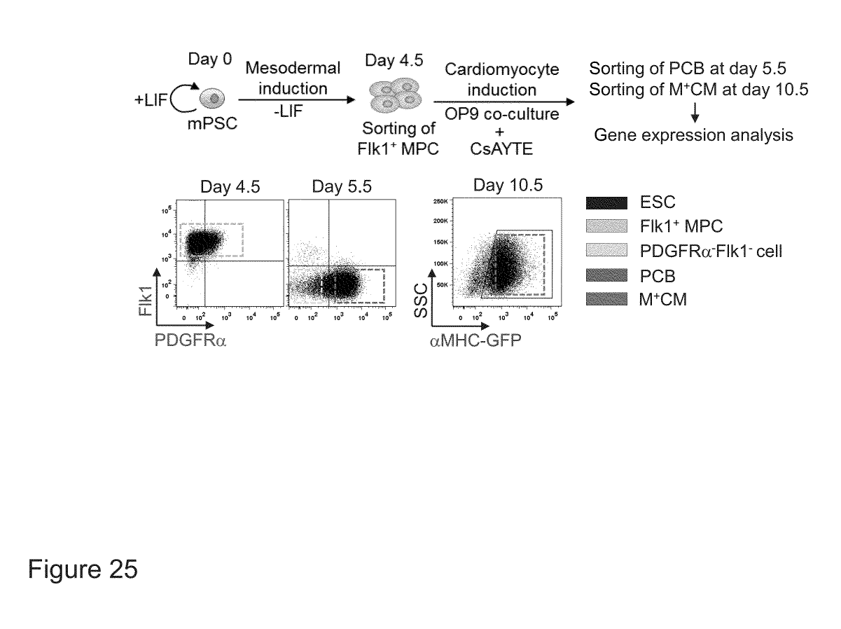

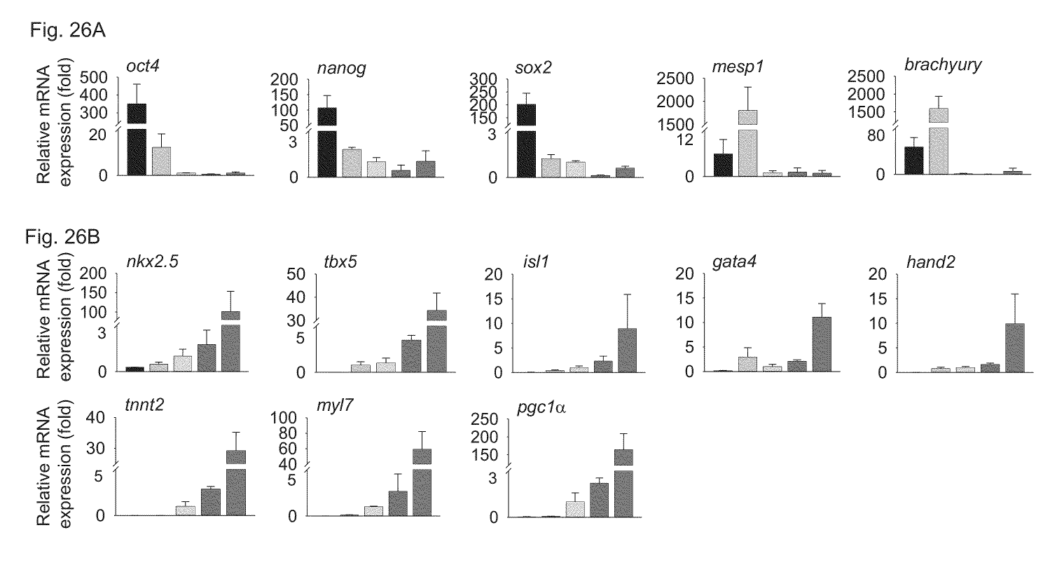

Next, to further delineate molecular properties of PCBs, we sorted out cells, analyzed cardiac-related gene expressions, and compared them with ESCs, Flk1⁺ MPCs, PDGFRα⁻Flk1⁻ cells, and M⁺CMs (FIG. 25). PCBs did not express pluripotent genes, such as oct4, nanog, and sox2 and mesodermal genes, including mesp1 and brachyury (FIG. 26A). However, expression levels of cardiac related transcription factors, such as nkx2.5, tbx5 and isl1, but not gata4 and hand2, were increased compared to others, while showing lower levels in contrast to M⁺CMs. Cardiac specific genes, including tnnt2 and myl7, and mitochondrial biogenesis markers, such as pgc1α, also showed similar patterns (FIG. 26B). These data suggest that PCBs are at an intermediate state between MPCs and differentiated cardiomyocytes.

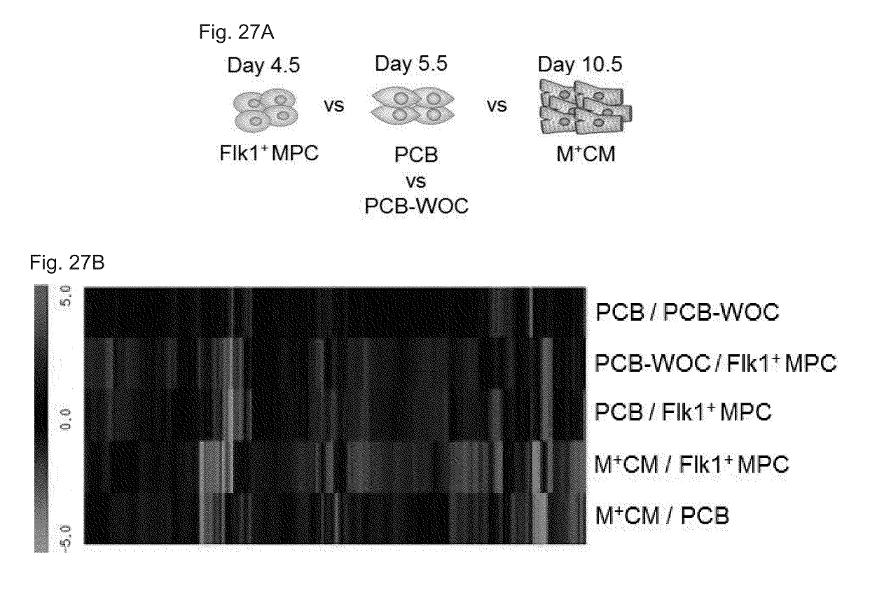

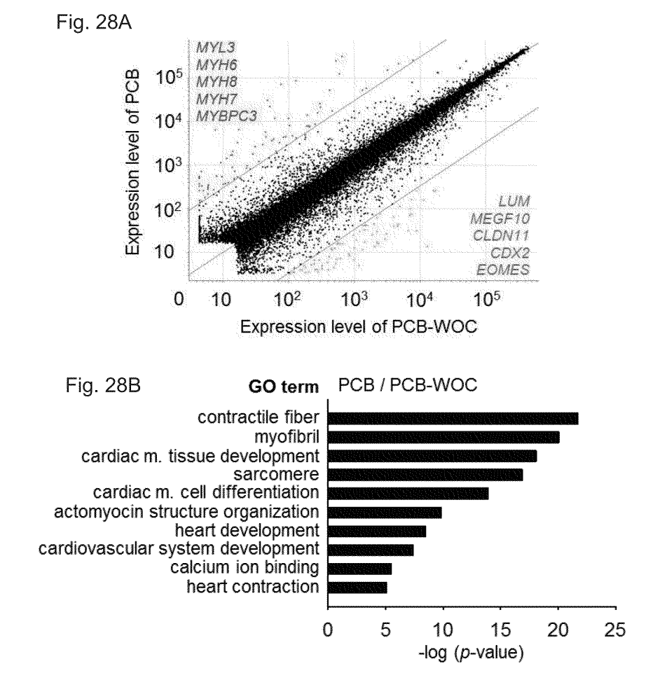

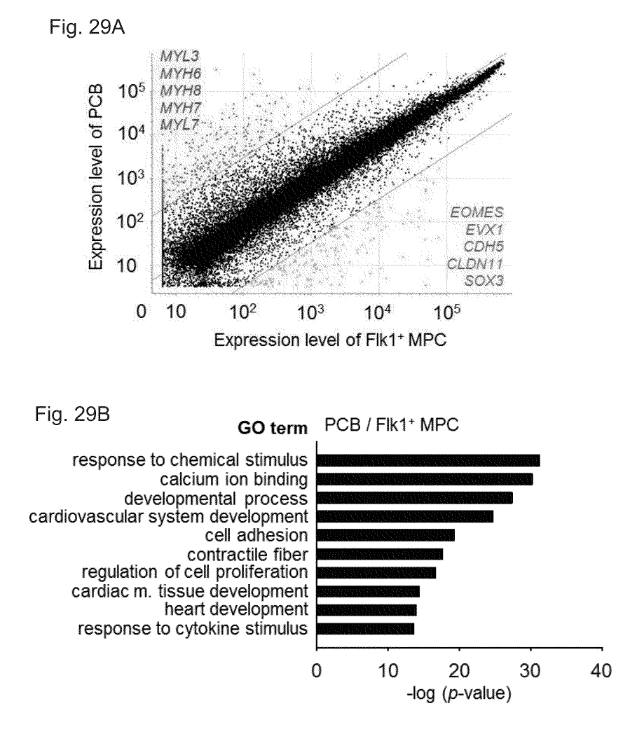

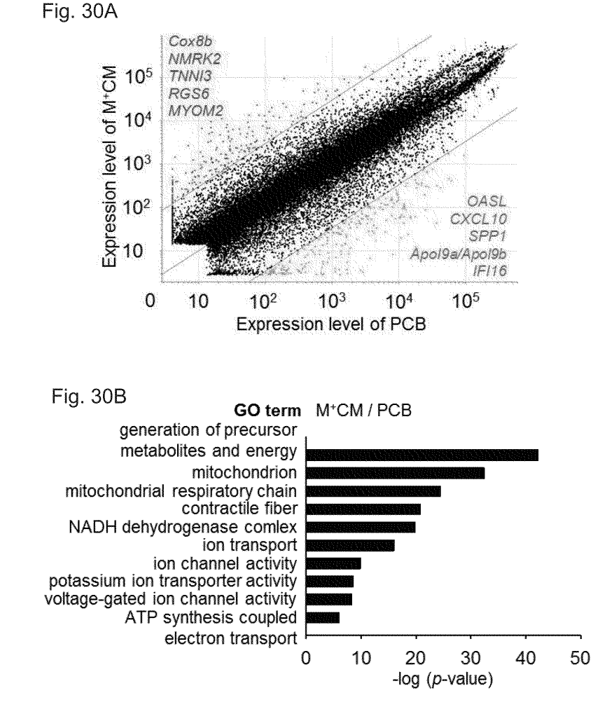

Finally, to elucidate genome-wide associated characteristics of PCBs, microarray analysis was performed and compared with PCBs without CsAYTE incubation (hereafter referred to as “PCBs-WOC”), Flk1⁺ MPCs, and M⁺CMs (FIGS. 27A and 27B). Comparison of PCBs and PCBs-WOC at day 5.5 identified 163 differently (≧30-fold) expressed transcripts (FIG. 28A). Gene ontology analysis revealed a significant increase of genes related to heart and muscle development in PCBs compared to PCBs-WOC (FIG. 28B). Moreover, comparison of PCBs and Flk1⁺ MPCs identified 558 differently (≧30-fold) expressed transcripts (FIG. 29A). Gene ontology analysis also showed that PCBs highly expressed genes belonging to chemical and cytokine stimulus, cardiovascular system development, and cell adhesion and proliferation compared to Flk1⁺ MPCs (FIG. 29B). Notably, gene expression profiles and ontology of M⁺CMs revealed a profound up-regulation of genes related to mitochondrial function and metabolism and ion channel activity compared to PCBs (FIGS. 30A and 30B).

Collectively, these results indicate that PCBs could be characterized as proliferating cardiac lineage-committed cells, which are still in a morphologically and functionally immature state compared to differentiated cardiomyocytes.

### Example 2.4

### Implantation of M⁺CMs, but not of PCBs, is Effective for Cardiac Regeneration after Acute MI

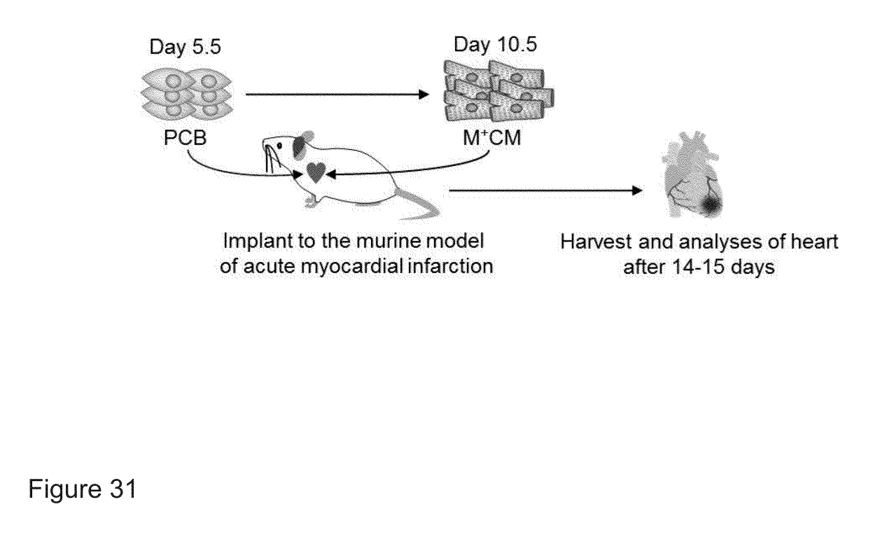

Based on our findings, we can generate an ample amount of functional M⁺CMs derived from PCBs for implantation. To investigate the regenerative potential of M⁺CMs, cells were sorted and implanted into the infarcted left ventricular myocardium after left anterior descending coronary artery ligation (FIG. 31) and compared with MI heart without implantation (hereafter called as “MI⁰”).

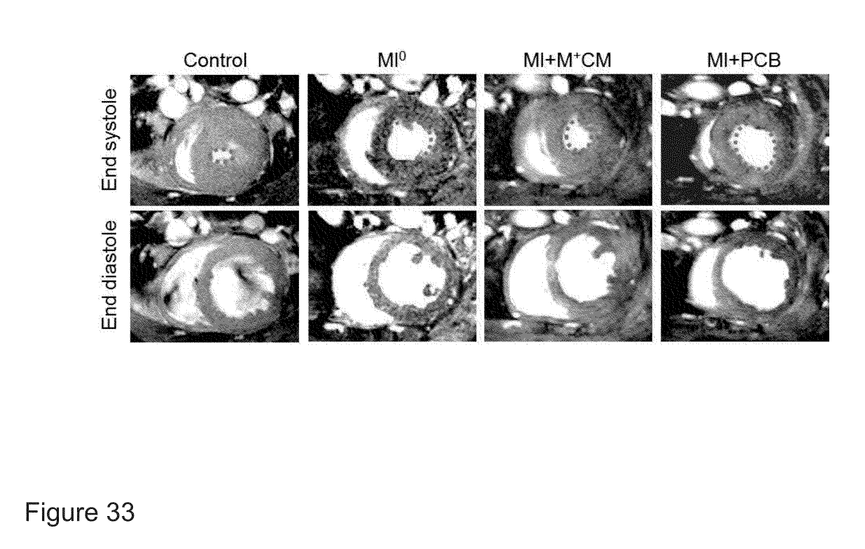

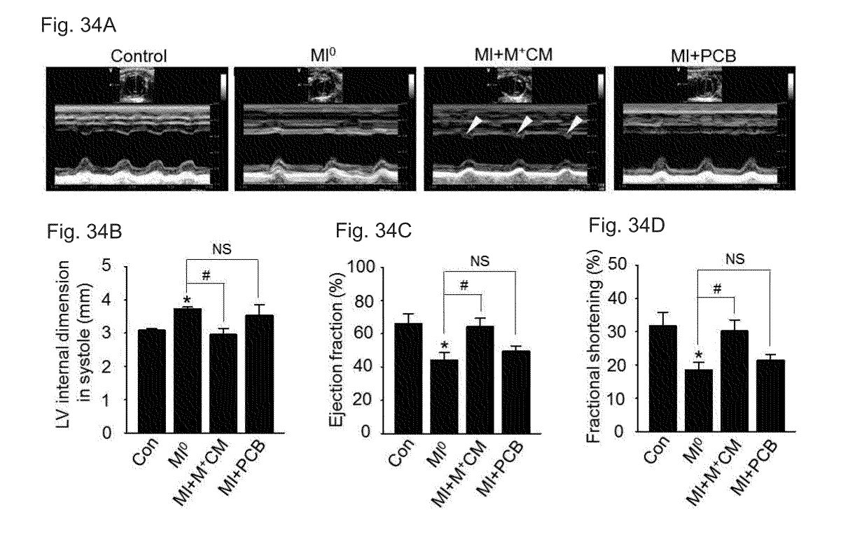

First, to evaluate functional recovery in infarcted hearts after cell implantation, we performed cardiac magnetic resonance imaging (MRI) and transthoracic echocardiography (TTE) 14 days after implantation. Compared to MI⁰, anterior and septal regional wall motion improved in MI of M⁺CM-implanted hearts (hereafter called as “MI+M⁺CM”). Moreover, MI+M⁺CM exhibited 59.4% reduction in the left ventricular end systolic area measured by cardiac MRI compared to MI⁰ (FIG. 33). Similarly, left ventricular internal dimension in systole measured by TTE of MI+M⁺CM decreased by 20% compared to that of MI⁰ (FIGS. 34A and 34B). MI+M⁺CM also showed significant improvements of left ventricular wall motion (white arrow heads) and systolic functional parameters, which are ejection fraction (20.2%) and fractional shortening (11.7%) (FIGS. 34A, C and D) compared to those of MI⁰. These findings indicate that M⁺CMs derived from PCBs have beneficial effects in the functional recovery of infarcted hearts.

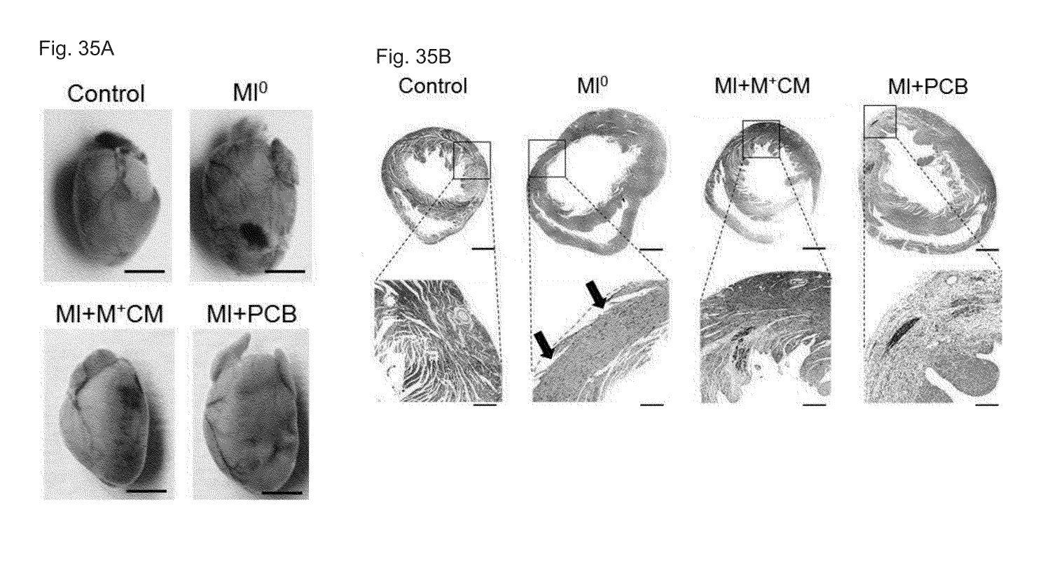

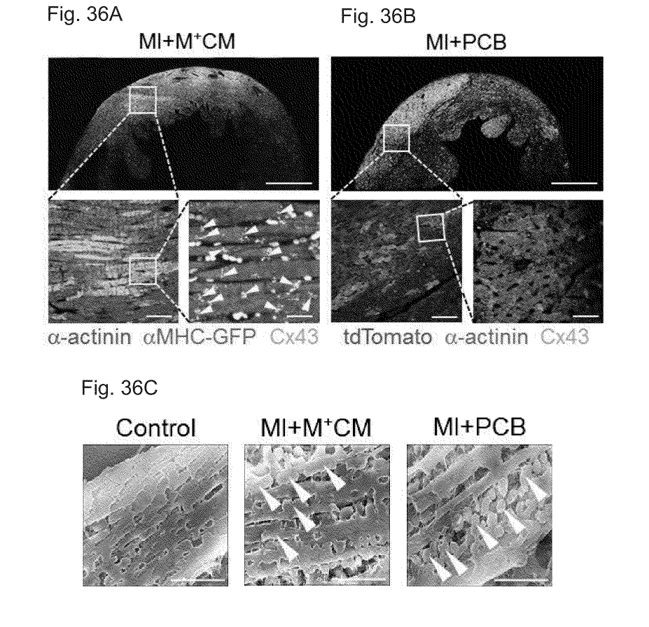

Next, to confirm whether the implanted cells were properly engrafted to the infarcted myocardium, we performed histological and immunohistochemistry analysis 15 days after implantation. Consistent with functional assays, the gross size of MI+M⁺CM was smaller than MI⁰ (FIG. 35A). Hematoxylin and eosin (H&E) staining showed that MI⁰ have several tissue defects (black arrows) and thinner ventricular walls, while ventricular walls of MI+M⁺CMs were preserved compared to those of MI⁰ (FIG. 35B). Importantly, detailed histological analyses showed co-localization and alignment of implanted M⁺CMs along α-actinin⁺ host cardiomyocytes, integrated via well-defined connexin43⁺ gap junctions (white arrow heads in FIG. 36A). Consistently, scanning electron microscopy images further confirmed the successful engraftment of implanted cardiomyocytes (white arrow heads in FIG. 36C), which were well integrated and aligned along host cardiomyocytes.

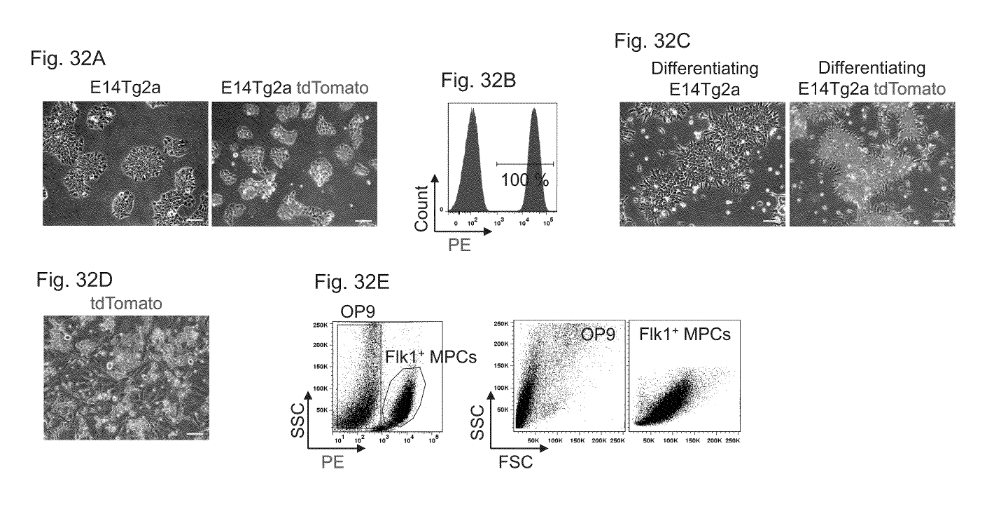

Although PCBs are relatively immature with limited contractile and electrical abilities compared to M⁺CMs, we thought that the PCBs may further differentiate and integrate into the host myocardium, and eventually provide regenerative potential in the infarcted heart (FIG. 31). To test our hypothesis, we implanted and traced the PCBs having tdTomato fluorescence (FIGS. 32A-E) in the infarcted heart. However, PCBs did not show significant functional and structural recovery in the acute MI model (FIGS. 33, 34A-D, 35A and 35B). Importantly, the engraftment pattern was quite different from implanted M⁺CMs. PCBs were not co-localized with α-actinin⁺ host cardiomyocytes and they were neither integrated nor aligned (FIG. 36B). Notably, PCBs did not form connexin43⁺ gap junction with host cardiomyocytes (FIG. 36B) and scanning electron microscopy images showed PCBs were not organized and aligned (white arrowheads in FIG. 36C). Thus, immature PCBs could not provide structural and functional regeneration of the infarcted heart. These data indicate that the differentiation status of cardiomyocytes derived from PSCs is critical for successful cell-mediated recovery of damaged hearts.

Taken together, CsAYTE promotes the differentiation of functional cardiomyocytes through massive expansion of PCBs. Moreover, implantation of PCB-derived functional cardiomyocytes, not immature PCBs, has regenerative capacity to rescue infarcted myocardium (FIGS. 37A and 37B). Therefore, CsAYTE is a powerful combination to generate ample amounts of differentiated cardioblasts for regeneration of damaged hearts.

### REFERENCES

Addis, R. C., and Epstein, J. A. (2013). Induced regeneration—the progress and promise of direct reprogramming for heart repair. Nature medicine 19, 829-836.
Ban, K., Wile, B., Kim, S., Park, H. J., Byun, J., Cho, K. W., Saafir, T., Song, M. K., Yu, S. P., Wagner, M., et al. (2013). Purification of cardiomyocytes from differentiating pluripotent stem cells using molecular beacons that target cardiomyocyte-specific mRNA. Circulation 128, 1897-1909.
Bondue, A., Tannler, S., Chiapparo, G., Chabab, S., Ramialison, M., Paulissen, C., Beck, B., Harvey, R., and Blanpain, C. (2011). Defining the earliest step of cardiovascular progenitor specification during embryonic stem cell differentiation. J Cell Biol 192, 751-765.
Burridge, P. W., Keller, G., Gold, J. D., and Wu, J. C. (2012). Production of de novo cardiomyocytes: human pluripotent stem cell differentiation and direct reprogramming. Cell stem cell 10, 16-28.
Cai, W., Guzzo, R. M., Wei, K., Willems, E., Davidovics, H., and Mercola, M. (2012). A Nodal-to-TGFbeta cascade exerts biphasic control over cardiopoiesis. Circulation research 111, 876-881.
Cao, N., Liu, Z., Chen, Z., Wang, J., Chen, T., Zhao, X., Ma, Y., Qin, L., Kang, J., Wei, B., et al. (2012). Ascorbic acid enhances the cardiac differentiation of induced pluripotent stem cells through promoting the proliferation of cardiac progenitor cells. Cell research 22, 219-236.
Chen, J. X., Krane, M., Deutsch, M. A., Wang, L., Rav-Acha, M., Gregoire, S., Engels, M. C., Rajarajan, K., Karra, R., Abel, E. D., et al. (2012). Inefficient reprogramming of fibroblasts into cardiomyocytes using Gata4, Mef2c, and Tbx5. Circulation research 111, 50-55.
Cho, H. J., Lee, C. S., Kwon, Y. W., Paek, J. S., Lee, S. H., Hur, J., Lee, E. J., Roh, T. Y., Chu, I. S., Leem, S. H., et al. (2010). Induction of pluripotent stem cells from adult somatic cells by protein-based reprogramming without genetic manipulation. Blood 116, 386-395.
Cho, S. W., Park, J. S., Heo, H. J., Park, S. W., Song, S., Kim, I., Han, Y. M., Yamashita, J. K., Youm, J. B., Han, J., et al. (2014). Dual modulation of the mitochondrial permeability transition pore and redox signaling synergistically promotes cardiomyocyte differentiation from pluripotent stem cells. Journal of the American Heart Association 3, e000693.
Chong, J. J., Yang, X., Don, C. W., Minami, E., Liu, Y. W., Weyers, J. J., Mahoney, W. M., Van Biber, B., Cook, S. M., Palpant, N. J., et al. (2014). Human embryonic-stem-cell-derived cardiomyocytes regenerate non-human primate hearts. Nature 510, 273-277.
Christoforou, N., Miller, R. A., Hill, C. M., Jie, C. C., McCallion, A. S., and Gearhart, J. D. (2008). Mouse ES cell-derived cardiac precursor cells are multipotent and facilitate identification of novel cardiac genes. The Journal of clinical investigation 118, 894-903.
Christoforou, N., Oskouei, B. N., Esteso, P., Hill, C. M., Zimmet, J. M., Bian, W., Bursac, N., Leong, K. W., Hare, J. M., and Gearhart, J. D. (2010). Implantation of mouse embryonic stem cell-derived cardiac progenitor cells preserves function of infarcted murine hearts. PloS one 5, e11536.
Dull, T., Zufferey, R., Kelly, M., Mandel, R. J., Nguyen, M., Trono, D., and Naldini, L. (1998). A third-generation lentivirus vector with a conditional packaging system. Journal of virology 72, 8463-8471.
Elliott, D. A., Braam, S. R., Koutsis, K., Ng, E. S., Jenny, R., Lagerqvist, E. L., Biben, C., Hatzistavrou, T., Hirst, C. E., Yu, Q. C., et al. (2011). NKX2-5(eGFP/w) hESCs for isolation of human cardiac progenitors and cardiomyocytes. Nature methods 8, 1037-1040.
Fujiwara, M., Yan, P., Otsuji, T. G., Narazaki, G., Uosaki, H., Fukushima, H., Kuwahara, K., Harada, M., Matsuda, H., Matsuoka, S., et al. (2011). Induction and enhancement of cardiac cell differentiation from mouse and human induced pluripotent stem cells with cyclosporine-A. PloS one 6, e16734.
Garbern, J. C., and Lee, R. T. (2013). Cardiac stem cell therapy and the promise of heart regeneration. Cell stem cell 12, 689-698.
Hirai, H., Ogawa, M., Suzuki, N., Yamamoto, M., Breier, G., Mazda, O., Imanishi, J., and Nishikawa, S. (2003). Hemogenic and nonhemogenic endothelium can be distinguished by the activity of fetal liver kinase (Flk)-1 promoter/enhancer during mouse embryogenesis. Blood 101, 886-893.
Hirata, H., Kawamata, S., Murakami, Y., Inoue, K., Nagahashi, A., Tosaka, M., Yoshimura, N., Miyamoto, Y., Iwasaki, H., Asahara, T., et al. (2007). Coexpression of platelet-derived growth factor receptor alpha and fetal liver kinase 1 enhances cardiogenic potential in embryonic stem cell differentiation in vitro. Journal of bioscience and bioengineering 103, 412-419.
Ieda, M., Fu, J. D., Delgado-Olguin, P., Vedantham, V., Hayashi, Y., Bruneau, B. G., and Srivastava, D. (2010). Direct reprogramming of fibroblasts into functional cardiomyocytes by defined factors. Cell 142, 375-386.
Joo, H. J., Choi, D. K., Lim, J. S., Park, J. S., Lee, S. H., Song, S., Shin, J. H., Lim, D. S., Kim, I., Hwang, K. C., et al. (2012). ROCK suppression promotes differentiation and expansion of endothelial cells from embryonic stem cell-derived Flk1(+) mesodermal precursor cells. Blood 120, 2733-2744.
Joo, H. J., Kim, H., Park, S. W., Cho, H. J., Kim, H. S., Lim, D. S., Chung, H. M., Kim, I., Han, Y. M., and Koh, G. Y. (2011). Angiopoietin-1 promotes endothelial differentiation from embryonic stem cells and induced pluripotent stem cells. Blood 118, 2094-2104.
Kattman, S. J., Huber, T. L., and Keller, G. M. (2006). Multipotent flk-1+ cardiovascular progenitor cells give rise to the cardiomyocyte, endothelial, and vascular smooth muscle lineages. Developmental cell 11, 723-732.
Kattman, S. J., Witty, A. D., Gagliardi, M., Dubois, N.C., Niapour, M., Hotta, A., Ellis, J., and Keller, G. (2011). Stage-specific optimization of activin/nodal and BMP signaling promotes cardiac differentiation of mouse and human pluripotent stem cell lines. Cell stem cell 8, 228-240.
Kawamura, M., Miyagawa, S., Miki, K., Saito, A., Fukushima, S., Higuchi, T., Kawamura, T., Kuratani, T., Daimon, T., Shimizu, T., et al. (2012). Feasibility, safety, and therapeutic efficacy of human induced pluripotent stem cell-derived cardiomyocyte sheets in a porcine ischemic cardiomyopathy model. Circulation 126, S29-37.
Kitamura, R., Takahashi, T., Nakajima, N., Isodono, K., Asada, S., Ueno, H., Ueyama, T., Yoshikawa, T., Matsubara, H., and Oh, H. (2007). Stage-specific role of endogenous Smad2 activation in cardiomyogenesis of embryonic stem cells. Circulation research 101, 78-87.
Kodama, H., Nose, M., Niida, S., and Nishikawa, S. (1994). Involvement of the c-kit receptor in the adhesion of hematopoietic stem cells to stromal cells. Experimental hematology 22, 979-984.
Laflamme, M. A., and Murry, C. E. (2011). Heart regeneration. Nature 473, 326-335.
Liu, F., Kang, I., Park, C., Chang, L. W., Wang, W., Lee, D., Lim, D. S., Vittet, D., Nerbonne, J. M., and Choi, K. (2012). ER71 specifies Flk-1+ hemangiogenic mesoderm by inhibiting cardiac mesoderm and Wnt signaling. Blood 119, 3295-3305.
Loffredo, F. S., Steinhauser, M. L., Gannon, J., and Lee, R. T. (2011). Bone marrow-derived cell therapy stimulates endogenous cardiomyocyte progenitors and promotes cardiac repair. Cell stem cell 8, 389-398.
Mummery, C. L., Zhang, J., Ng, E. S., Elliott, D. A., Elefanty, A. G., and Kamp, T. J. (2012). Differentiation of human embryonic stem cells and induced pluripotent stem cells to cardiomyocytes: a methods overview. Circulation research 111, 344-358.
Nelson, T. J., Faustino, R. S., Chiriac, A., Crespo-Diaz, R., Behfar, A., and Terzic, A. (2008). CXCR4+/FLK-1+ biomarkers select a cardiopoietic lineage from embryonic stem cells. Stem cells 26, 1464-1473.
Passier, R., van Laake, L. W., and Mummery, C. L. (2008). Stem-cell-based therapy and lessons from the heart. Nature 453, 322-329.
Qian, L., Huang, Y., Spencer, C. I., Foley, A., Vedantham, V., Liu, L., Conway, S. J., Fu, J. D., and Srivastava, D. (2012). In vivo reprogramming of murine cardiac fibroblasts into induced cardiomyocytes. Nature 485, 593-598.
Rompani, S. B., and Cepko, C. L. (2008). Retinal progenitor cells can produce restricted subsets of horizontal cells. Proceedings of the National Academy of Sciences of the United States of America 105, 192-197.
Ryan, T., Shelton, M., Lambert, J. P., Malecova, B., Boisvenue, S., Ruel, M., Figeys, D., Puri, P. L., and Skerjanc, I. S. (2013). Myosin phosphatase modulates the cardiac cell fate by regulating the subcellular localization of Nkx2.5 in a Wnt/Rho-associated protein kinase-dependent pathway. Circulation research 112, 257-266.
Scavone, A., Capilupo, D., Mazzocchi, N., Crespi, A., Zoia, S., Campostrini, G., Bucchi, A., Milanesi, R., Baruscotti, M., Benedetti, S., et al. (2013). Embryonic stem cell-derived CD166+ precursors develop into fully functional sinoatrial-like cells. Circulation research 113, 389-398.
Segers, V. F., and Lee, R. T. (2008). Stem-cell therapy for cardiac disease. Nature 451, 937-942.
Shiba, Y., Fernandes, S., Zhu, W. Z., Filice, D., Muskheli, V., Kim, J., Palpant, N.J., Gantz, J., Moyes, K. W., Reinecke, H., et al. (2012). Human ES-cell-derived cardiomyocytes electrically couple and suppress arrhythmias in injured hearts. Nature 489, 322-325.
Son, J. Y., Park, S. Y., Kim, S. J., Lee, S. J., Park, S. A., Kim, M. J., Kim, S. W., Kim, D. K., Nam, J. S., and Sheen, Y. Y. (2014). EW-7197, a novel ALK-5 kinase inhibitor, potently inhibits breast to lung metastasis. Molecular cancer therapeutics 13, 1704-1716.
Song, K., Nam, Y. J., Luo, X., Qi, X., Tan, W., Huang, G. N., Acharya, A., Smith, C. L., Tallquist, M. D., Neilson, E. G., et al. (2012). Heart repair by reprogramming non-myocytes with cardiac transcription factors. Nature 485, 599-604.
Soonpaa, M. H., Rubart, M., and Field, L. J. (2013). Challenges measuring cardiomyocyte renewal. Biochimica et biophysica acta 1833, 799-803.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Uosaki, H., Fukushima, H., Takeuchi, A., Matsuoka, S., Nakatsuji, N., Yamanaka, S., and Yamashita, J. K. (2011). Efficient and scalable purification of cardiomyocytes from human embryonic and induced pluripotent stem cells by VCAM1 surface expression. PloS one 6, e23657.
Verma, V., Purnamawati, K., Manasi, and Shim, W. (2013). Steering signal transduction pathway towards cardiac lineage from human pluripotent stem cells: a review. Cell Signal 25, 1096-1107.
Willems, E., Cabral-Teixeira, J., Schade, D., Cai, W., Reeves, P., Bushway, P. J., Lanier, M., Walsh, C., Kirchhausen, T., Izpisua Belmonte, J. C., et al. (2012). Small molecule-mediated TGF-beta type II receptor degradation promotes cardiomyogenesis in embryonic stem cells. Cell stem cell 11, 242-252.
Yamashita, J., Itoh, H., Hirashima, M., Ogawa, M., Nishikawa, S., Yurugi, T., Naito, M., Nakao, K., and Nishikawa, S. (2000). Flk1-positive cells derived from embryonic stem cells serve as vascular progenitors. Nature 408, 92-96.
Yamashita, J. K., Takano, M., Hiraoka-Kanie, M., Shimazu, C., Peishi, Y., Yanagi, K., Nakano, A., Inoue, E., Kita, F., and Nishikawa, S. (2005). Prospective identification of cardiac progenitors by a novel single cell-based cardiomyocyte induction. FASEB journal: official publication of the Federation of American Societies for Experimental Biology 19, 1534-1536.
Yan, P., Nagasawa, A., Uosaki, H., Sugimoto, A., Yamamizu, K., Teranishi, M., Matsuda, H., Matsuoka, S., Ikeda, T., Komeda, M., et al. (2009). Cyclosporine-A potently induces highly cardiogenic progenitors from embryonic stem cells. Biochem Biophys Res Commun 379, 115-120.
Yang, L., Soonpaa, M. H., Adler, E. D., Roepke, T. K., Kattman, S. J., Kennedy, M., Henckaerts, E., Bonham, K., Abbott, G. W., Linden, R. M., et al. (2008). Human cardiovascular progenitor cells develop from a KDR+ embryonic-stem-cell-derived population. Nature 453, 524-528.
Zaruba, M. M., Soonpaa, M., Reuter, S., and Field, L. J. (2010). Cardiomyogenic potential of C-kit(+)-expressing cells derived from neonatal and adult mouse hearts. Circulation 121, 1992-2000.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method for inducing pluripotent cell to form cardioblast comprising contacting the pluripotent cell with an effective amount of a composition comprising inhibitor of mitochondrial permeability transition pore (mPTP), Rho-associated protein kinase (ROCK) inhibitor, antioxidant, or activin A receptor type II-like kinase (ALK5) inhibitor compound so as to form cardioblast.

2. The method according to claim 1, wherein the composition comprises any two of inhibitor of mitochondrial permeability transition pore (mPTP), ROCK inhibitor, antioxidant, or activin A receptor type II-like kinase (ALK5) inhibitor compound.

3. The method according to claim 1, wherein the composition comprises any three of inhibitor of mitochondrial permeability transition pore (mPTP), ROCK inhibitor, antioxidant, or activin A receptor type II-like kinase (ALK5) inhibitor compound.

4. The method according to claim 1, wherein the composition comprises all four of inhibitor of mitochondrial permeability transition pore (mPTP), ROCK inhibitor, antioxidant, or activin A receptor type II-like kinase (ALK5) inhibitor compound.

5. The method according to claim 1, wherein the composition further comprises Wnt signaling inhibitor compound.

6. The method according to claim 1, wherein the inhibitor of mitochondrial permeability transition pore (mPTP) is cyclosporine.

7. The method according to claim 6, wherein the cyclosporine is Cyclosporine-A.

8. The method according to claim 1, wherein the ROCK inhibitor is RKI, RKI-II, siRNA-ROCK1, si-RNA-ROCK2 or chemical compound specific to ROCK1 or ROCK2.

9. The method according to claim 8, wherein the chemical compound specific to ROCK1 or ROCK2 is [(+)-(R)-trans-4-(1-aminoethyl)-N-(4-pyridyl) cyclohexanecarboxamide dihydrochloride] (Y27362).

10. The method according to claim 1, wherein the antioxidant is Trolox (6-hydroxy-2,5,7,8-tetramethylchroman-2-carboxylic acid).

11. The method according to claim 1, activin A receptor type II-like kinase (ALK5)inhibitor is N-[[4-([1,2,4]triazolo[1,5-a]pyridin-6-yl)-5-(6-methylpyridin-2-yl)-1H-imidazol-2-yl]methyl]-2-fluoroaniline (EW-7197).

12. The method according to claim 1, which is conducted in vitro.

13. The method according to claim 1, which is conducted in vivo.

14. The method according to claim 1, wherein the pluripotent cell is embryonic stem cell (ESC).

15. The method according to claim 1, wherein the pluripotent cell is mesodermal stem cell (MSC).

16. The method according to claim 1, wherein the pluripotent cell is a mammalian cell.

17. The method according to claim 16, wherein the mammalian cell is mouse or human cell.

18. The method according to claim 15, wherein phenotype for the mesodermal precursor cell is Flk1⁺.

19. The method according to claim 1, wherein phenotype of the cardioblast is characterized by PDGFRα⁺Flk1⁻.

20. A method of generating cardiomyocytes comprising allowing the obtained cardioblast according to claim 1 to proliferate with or without contact with the composition according to claim 1, to result in cardiomyocytes.

21. A method for generating and expanding differentiated cardiomyocytes comprising contacting a pluripotent cell with an effective amount of a composition comprising inhibitor of mitochondrial permeability transition pore (mPTP), Rho-associated protein kinase (ROCK) inhibitor, antioxidant, or activin A receptor type II-like kinase (ALK5) inhibitor compound so as to form cardiomyocyte.

22. The method according to claim 21, wherein the composition comprises any two of inhibitor of mitochondrial permeability transition pore (mPTP), ROCK inhibitor, antioxidant, or activin A receptor type II-like kinase (ALK5) inhibitor compound.

23. The method according to claim 21, wherein the composition comprises any three of inhibitor of mitochondrial permeability transition pore (mPTP), ROCK inhibitor, antioxidant, or activin A receptor type II-like kinase (ALK5) inhibitor compound.

24. The method according to claim 21, wherein the composition comprises all four of inhibitor of mitochondrial permeability transition pore (mPTP), ROCK inhibitor, antioxidant, or activin A receptor type II-like kinase (ALK5) inhibitor compound.

25. The method according to claim 21, wherein the composition further comprises Wnt signaling inhibitor compound.

26. The method according to claim 21, wherein the inhibitor of mitochondrial permeability transition pore (mPTP) is cyclosporine.

27. The method according to claim 26, wherein the cyclosporine is Cyclosporine A.

28. The method according to claim 21, wherein the ROCK inhibitor is RKI, RKI-II, siRNA-ROCK1, si-RNA-ROCK2 or chemical compound specific to ROCK1 or ROCK2.

29. The method according to claim 28, wherein the chemical compound specific to ROCK1 or ROCK2 is [(+)-(R)-trans-4-(1-aminoethyl)-N-(4-pyridyl) cyclohexanecarboxamide dihydrochloride] (Y27362).

30. The method according to claim 21, wherein the antioxidant is Trolox (6-hydroxy-2,5,7,8-tetramethylchroman-2-carboxylic acid).

31. The method according to claim 21, activin A receptor type II-like kinase (ALK5)inhibitor is N-[[4-([1,2,4]triazolo[1,5-a]pyridin-6-yl)-5-(6-methylpyridin-2-yl)-1H-imidazol-2-yl]methyl]-2-fluoroaniline (EW-7197).

32. The method according to claim 21, which is conducted in vitro.

33. The method according to claim 21, which is conducted in vivo.

34. The method according to claim 21, wherein the pluripotent cell is embryonic stem cell (ESC).

35. The method according to claim 21, wherein the pluripotent cell is mesodermal stem cell (MSC).

36. The method according to claim 21, wherein the pluripotent cell is a mammalian cell.

37. The method according to claim 36, wherein the mammalian cell is mouse or human cell.

38. The method according to claim 35, wherein phenotype for the mesodermal precursor cell is Flk1⁺.

39. The method according to claim 21, comprising withdrawing contact with pluripotent cell when cardioblast is formed.

40. The method according to claim 39, wherein phenotype of the cardioblast is characterized by PDGFRα⁺Flk1⁻.

41. A method of regenerating a portion of a heart in a subject comprising:
(i) generating and expanding differentiated cardiomyocytes comprising contacting a pluripotent cell with an effective amount of a composition comprising inhibitor of mitochondrial permeability transition pore (mPTP), Rho-associated protein kinase (ROCK) inhibitor, antioxidant, or activin A receptor type II-like kinase (ALK5) inhibitor compound so as to form cardiomyocyte; and
(ii) administering to the subject the cardiomyocyte obtained in step (i) so as to expand cardiomyocytes in the heart.

42. The method according to claim 41, wherein heart is damaged heart.

43. The method according to claim 42, wherein the damaged heart is cardiomyopathy, myocardial infarction, acute myocardial infarction, chronic heart failure, ischemic and dilated cardiomyopathy, sick sinus syndrome, or congenital heart disease.

44. The method according to claim 41, wherein the composition comprises any two of inhibitor of mitochondrial permeability transition pore (mPTP), ROCK inhibitor, antioxidant, or activin A receptor type II-like kinase (ALK5) inhibitor compound.

45. The method according to claim 41, wherein the composition comprises any three of inhibitor of mitochondrial permeability transition pore (mPTP), ROCK inhibitor, antioxidant, or activin A receptor type II-like kinase (ALK5) inhibitor compound.

46. The method according to claim 41, wherein the composition comprises all four of inhibitor of mitochondrial permeability transition pore (mPTP), ROCK inhibitor, antioxidant, or activin A receptor type II-like kinase (ALK5) inhibitor compound.

47. The method according to claim 41, wherein the composition further comprises Wnt signaling inhibitor compound.

48. The method according to claim 41, wherein the mPTP inhibitor is cyclosporine.

49. The method according to claim 48, wherein the cyclosporine is Cyclosporine A.

50. The method according to claim 41, wherein the ROCK inhibitor is RKI, RKI-II, siRNA-ROCK1, si-RNA-ROCK2 or chemical compound specific to ROCK1 or ROCK2.

51. The method according to claim 50, wherein the chemical compound specific to ROCK1 or ROCK2 is [(+)-(R)-trans-4-(1-aminoethyl)-N-(4-pyridyl) cyclohexanecarboxamide dihydrochloride] (Y27362).

52. The method according to claim 41, wherein the antioxidant is Trolox (6-hydroxy-2,5,7,8-tetramethylchroman-2-carboxylic acid).

53. The method according to claim 41, activin A receptor type II-like kinase (ALK5)inhibitor is N-[[4-([1,2,4]triazolo[1,5-a]pyridin-6-yl)-5-(6-methylpyridin-2-yl)-1H-imidazol-2-yl]methyl]-2-fluoroaniline (EW-7197).

54. The method according to claim 41, which is conducted in vitro.

55. The method according to claim 41, which is conducted in vivo.

56. The method according to claim 41, wherein the pluripotent cell is embryonic stem cell (ESC).

57. The method according to claim 41, wherein the pluripotent cell is mesodermal stem cell (MSC).

58. The method according to claim 41, wherein the pluripotent cell is a mammalian cell.

59. The method according to claim 58, wherein the mammalian cell is mouse or human cell.

60. The method according to claim 57, wherein phenotype for the mesodermal precursor cell is Flk1⁺.

61. The method according to claim 41, step (i), comprising withdrawing contact with pluripotent cell when cardioblast is formed.

62. The method according to claim 61, wherein phenotype of the cardioblast is characterized by PDGFRα⁺Flk1⁻.

63. A method of regenerating a portion of a heart in a subject comprising:
(i) generating and expanding cardioblast comprising contacting a pluripotent cell with an effective amount of a composition comprising inhibitor of mitochondrial permeability transition pore (mPTP), Rho-associated protein kinase (ROCK) inhibitor, antioxidant, or activin A receptor type II-like kinase (ALK5) inhibitor compound so as to form cardioblast; and
(ii) administering to the subject the cardioblast obtained in step (i) so as to differentiate into cardiomyocytes in the heart.

64. The method according to claim 63, wherein the heart is damaged heart.

65. The method according to claim 64, wherein the damage to the heart is cardiomyopathy, myocardial infarction, acute myocardial infarction, chronic heart failure, ischemic and dilated cardiomyopathy, sick sinus syndrome, or congenital heart disease.


SEQUENCE LISTING (30 sequences)

SEQ ID NO: 1 - DNA, 23 bp, Artificial Sequence
  Forward primer Mouse oct3/4
tctttccaccaggcccccggctc

SEQ ID NO: 2 - DNA, 23 bp, Artificial Sequence
  Reverse primer Mouse oct3/4
tgcgggcggacatggggagatcc

SEQ ID NO: 3 - DNA, 24 bp, Artificial Sequence
  Forward primer Mouse nanog
agggtctgctactgagatgctctg

SEQ ID NO: 4 - DNA, 24 bp, Artificial Sequence
  Reverse primer Mouse nanog
caaccactggtttttctgccaccg

SEQ ID NO: 5 - DNA, 23 bp, Artificial Sequence
  Forward primer Mouse sox2
tagagctagactccgggcgatga

SEQ ID NO: 6 - DNA, 23 bp, Artificial Sequence
  Reverse primer Mouse sox2
ttgccttaaacaagaccacgaaa

SEQ ID NO: 7 - DNA, 25 bp, Artificial Sequence
  Forward primer Mouse mesp1
ccatcgttcctgtacgcagaaacag

SEQ ID NO: 8 - DNA, 25 bp, Artificial Sequence
  Reverse primer Mouse mesp1
agacagggtgacaatcatccgttgc

SEQ ID NO: 9 - DNA, 25 bp, Artificial Sequence
  Forward primer Mouse brachyury
ctatgctcatcggaacagctctcca

SEQ ID NO: 10 - DNA, 24 bp, Artificial Sequence
  Reverse primer Mouse brachyury
ctcacagaccagagactgggatac

SEQ ID NO: 11 - DNA, 25 bp, Artificial Sequence
  Forward primer Mouse gata4
ccctctttgtcattcttcgctggag

SEQ ID NO: 12 - DNA, 26 bp, Artificial Sequence
  Reverse primer Mouse gata4
gatttgcggttgctccagaaatcgtg

SEQ ID NO: 13 - DNA, 26 bp, Artificial Sequence
  Forward primer Mouse nkx2.5
cacgcctttctcagtcaaagacatcc

SEQ ID NO: 14 - DNA, 24 bp, Artificial Sequence
  Reverse primer Mouse nkx2.5
ctgggaaagcaggagagcacttgg

SEQ ID NO: 15 - DNA, 24 bp, Artificial Sequence
  Forward primer Mouse tbx5
cgcctctggagcctgattccaaag

SEQ ID NO: 16 - DNA, 24 bp, Artificial Sequence
  Reverse primer Mouse tbx5
gtgcccacttcgtggaacttcagc

SEQ ID NO: 17 - DNA, 23 bp, Artificial Sequence
  Forward primer Mouse isl1
agaccctctcagtcccttgcatc

SEQ ID NO: 18 - DNA, 25 bp, Artificial Sequence
  Reverse primer Mouse isl1
catctccactagttgctccttcatg

SEQ ID NO: 19 - DNA, 23 bp, Artificial Sequence
  Forward primer Mouse hand2
caagatcaagacactgcgcctgg

SEQ ID NO: 20 - DNA, 24 bp, Artificial Sequence
  Reverse primer Mouse hand2
tcgttgctgctcactgtgcttttc

SEQ ID NO: 21 - DNA, 24 bp, Artificial Sequence
  Forward Primer Mouse tnnt2
gacctgtgtgcagtccctgttcag

SEQ ID NO: 22 - DNA, 24 bp, Artificial Sequence
  Reverse Primer Mouse tnnt2
cttgctcgtcctcctcttcttcac

SEQ ID NO: 23 - DNA, 25 bp, Artificial Sequence
  Forward primer Mouse myl7
atcagacctgaaggagacctattcc

SEQ ID NO: 24 - DNA, 23 bp, Artificial Sequence
  Reverse primer Mouse myl7
aaggcactcaggatggcttcctc

SEQ ID NO: 25 - DNA, 19 bp, Artificial Sequence
  Forward primer Mouse pgc1
gcgccgtgtgatttacgtt

SEQ ID NO: 26 - DNA, 23 bp, Artificial Sequence
  Reverse primer Mouse pgc1
aaaacttcaaagcggtctctcaa

SEQ ID NO: 27 - DNA, 24 bp, Artificial Sequence
  Forward primer Mouse connexin43
gtgtctgtgcccacactcctgtac

SEQ ID NO: 28 - DNA, 24 bp, Artificial Sequence
  Reverse primer Mouse connexin43
ctcagcaggccacctctcatcttc

SEQ ID NO: 29 - DNA, 20 bp, Artificial Sequence
  Forward Primer Mouse beta actin
gctcttttccagccttcctt

SEQ ID NO: 30 - DNA, 20 bp, Artificial Sequence
  Reverse primer Mouse beta actin
cttctgcatcctgtcagcaa